(19) United States
(12) Patent Application Publication
(10) Pub. No.: US 2024/0161291 A1
(43) Pub. Date: May 16, 2024

(54) Imaging-Enabled Bioreactor for Ex Vivo Human Airway Tissues

(71) Applicants: The Trustees of The Stevens Institute of Technology, Hoboken, NJ (US); The Trustees of the Columbia University in the City of New York, New York, NY (US)

(72) Inventors: Jinho Kim, Jersey City, NJ (US); Seyed Mohammad Mir, Hoboken, NJ (US); Jiawen Chen, Hoboken, NJ (US); Gordana Vunjak-Novakovi, New York, NY (US); Meghan Pinezich, New York, NY (US); Maria Hudock, New York, NY (US)

(21) Appl. No.: 18/505,986

(22) Filed: Nov. 9, 2023

Related U.S. Application Data
(60) Provisional application No. 63/383,239, filed on Nov. 10, 2022.

(51) Int. Cl. G06T 7/00; C12M 3/00; G16H 50/50

(52) U.S. Cl. CPC G06T 7/0012; C12M 21/08; G16H 50/50; G06T 2207/30004

(57) ABSTRACT

A bioreactor is disclosed that can be used to study living tissues (e.g., lung tissues) in an environment that mimics the natural environment. Pressure, flow and force modules enable imitation of in vivo conditions. An integrated imaging module allows for the airway tissues and cells of interest to be visualized and monitored continuously and non-destructively at the single-cell level. A method of use of the created systems can be utilized to quantify mucociliary fluid movements across the luminal surface of in vitro-cultured human airway tissue via in situ particle tracking and analysis is also disclosed. Another method of use of systems created in accordance with an embodiment of the present invention is to generate in vitro-cultured human airway tissue with severely impaired mucociliary flow by depositing thick viscous mucus-mimetic fluid on to the airway lumen. Methods of de-epithelialization and replacement of living cells are also disclosed.

### CROSS-REFERENCE TO RELATED APPLICATION

This application claims priority to U.S. Provisional Patent Application Ser. No. 63/383,239 filed Nov. 10, 2022, the entire disclosure of which is incorporated herein by reference.

### STATEMENT REGARDING FEDERALLY SPONSORED RESEARCH

This invention was made with government support under EB027062 awarded by the National Institutes of Health and 2143620 awarded by the National Science Foundation. The government has certain rights in the invention.

### FIELD OF THE INVENTION

The invention relates to devices or systems that can allow real-time assessment of airway tissue regeneration, disease modeling, and drug testing using airway tissues obtained from human or large animal lung.

### BACKGROUND OF THE INVENTION

The human conducting airways are lined by the airway epithelium that mostly consists of multi-ciliated, club, goblet, and basal cells. These airway epithelial cells collectively create a protective biophysical barrier between the external environment and underlying tissues against inhaled harmful substances, such as pathogens, allergens, chemical gases, and/or particulates. The protective functions of the airway epithelium include mucociliary clearance, tight junction formation, and antimicrobial secretion. While serving as the first line of defense of the lung, the airway epithelium is the prime site for the initiation and progression of many devastating respiratory disorders, such as cystic fibrosis (CF), asthma, chronic obstructive pulmonary diseases (COPD), or primary ciliary dyskinesia (PCD).

There are commercially available in vitro airway mimics, such as the Airway Lung-Chip (Emulate, Inc.), and airway tissue bioreactors, such as InBreath 3D Bioreactor (Harvard Bioscience, Inc.). The Airway Lung-Chip can be used for modeling and studying pulmonary physiology and diseases in vitro. InBreath 3D Bioreactor can be used for seeding and culturing of different cell types onto de-cellularized airway tissues. Existing systems do not use actual airway tissues, do not recapitulate the complex biology and geometry of native airway tissues, and/or lack in real-time cell and tissue monitoring capability.

### SUMMARY OF THE INVENTION

None of the prior art devices are capable of fully recapitulating the intrinsic biophysical characteristics of native airway tissues. None of the devices can create user-defined breathing conditions to the cultured airway tissues, such as cough. Additionally, none of the devices can be used to simulate mucociliary impairment in vitro. On the other hand, the imaging-guided bioreactor system of the present invention allows the use of segments of airway tissues obtained from human or animal lung, provides near-physiologic microenvironments to the cultured cells and tissues, and is capable of visualizing and monitoring continuously and non-destructively the airway tissues and cells at the single-cell level. The present invention addresses the lack of commercial devices or systems that can allow real-time assessment of airway tissue regeneration, disease modeling, and drug testing using airway tissues obtained from human or large animal lung.

The present invention relates to an imaging-enabled bioreactor system that allows long-term in vitro cultivation, tissue engineering, and/or drug testing using a small segment of airway tissue isolated from human lung. The bioreactor is capable of visualization of the in vitro-cultured airway tissue at the cellular level during tissue manipulation and drug testing. The human airway bioreactor system of the present invention is combined with: i) an innovative in situ imaging modality for real-time tissue monitoring and ii) computer-controlled sensors and actuators for precise control of local tissue environment. This integrated system can allow generation of in vitro airway models that mimic diseased airway tissues, such as CF, asthma, COPD, or PCD, thereby allowing efficient investigations of pathogenesis, pathophysiology, or drug efficacy.

In an embodiment, the present invention comprises an airway tissue bioreactor system integrated with in-situ microscopic imaging modalities and computerized flow, pressure, and force control modules that can synergistically allow investigation of tissue regeneration, modeling of diseases, and evaluation of drug efficacy using in vitro-cultured airway tissues, such as trachea, bronchus, or bronchiole, obtained from human lung, under tightly regulated in vitro environments (FIG. 1; FIG. 2)

In another embodiment, the present invention involves a method of use of this system to controllably replace endogenous airway cells with exogenous airway primary or stem cells to create healthy or diseased in vitro human airway tissue models (FIG. 3).

In further embodiments of the present invention, a method of use of the inventive system allows for the selective removal of cellular components from in vitro-cultured human airway tissue via a combination of mechanical and chemical treatments (FIG. 3A; FIG. 4).

In a still further embodiment of the present invention, a method of use of the inventive system allows a user to rapidly and uniformly repopulate in vitro-cultured human airway tissue scaffold with exogenous cells via hydrogel-or culture medium-based cell delivery (FIG. 3B).

In yet another embodiment of the present invention, the inventive method can establish a system incorporating diseased cells and/or tissues as a model of disease in vitro (FIG. 3B).

In another embodiment of the present invention, a method of use of a system to develop bioengineered human airway tissues to replace all or part of a damaged lung through transplantation in vivo is enabled (FIG. 3C).

In further embodiments, a method of use of systems made in accordance with an embodiment of the present invention allows for the non-destructive and local monitoring of the removal of endogenous cells and distribution of exogenous cells within in vitro-cultured human airway tissues using an in situ microscopic fluorescent imaging modality (FIG. 5; FIGS. 6A-6E).

In alternate embodiments of the present invention, a method of use of the created systems can be utilized to quantify mucociliary fluid movements across the luminal surface of in vitro-cultured human airway tissue via in situ particle tracking and analysis (FIGS. 7A-7C).

Another method of use of systems created in accordance with an embodiment of the present invention is to generate in vitro-cultured human airway tissue with severely impaired mucociliary flow by depositing thick viscous mucus-mimetic fluid on to the airway lumen (FIG. 8, FIG. 9).

A further method of use of systems created in accordance with an embodiment of the present invention involves investigation into the effects of mechanical vibration and chemical treatment on removal of thick mucus layer from the inner lumen of in vitro-cultured human airway tissue (FIG. 3A, FIGS. 4A-4F, FIG. 9A). A further related method involves the use of such systems to create a coughing flow within in vitro-cultured human airway tissue by regulating the flowrate and pressure of airflow (FIG. 10).

A further method related to the use of systems created in accordance with an embodiment of the present invention is the study of therapeutics and drug delivery to the in vitro-cultured human airways including via liquid instillation and/or aerosolization (FIG. 11).

A not-necessarily final embodiment of the present invention entails a method of use of a created system to monitor effects of delivered drugs, gene therapies, and other therapeutics via in situ microscopic fluorescent imaging and other nondestructive reporters, such as bioluminescent imaging (FIG. 11).

The de-epithelialization method, cell replacement technique, and optical fiber-based imaging techniques of the present invention are applicable for in vitro disease modeling, investigations, and drug testing of various hollow or tubular tissues or organs in addition to just lung tissue. These include but are not limited to the intestines, blood vessels, esophagus, fallopian tubes, stomach, or bladder.

### BRIEF DESCRIPTION OF THE DRAWINGS

For a more complete understanding of the present invention, reference is made to the following detailed description of various embodiments considered in conjunction with the accompanying drawings, in which:

FIG. 1A is a schematic overview of an imaging-enabled bioreactor system and deepithelialization of ex vivo human airway tissues in accordance with an embodiment of the present invention;

FIG. 1B is a perspective view of the bioreactor system of FIG. 1A, showing an enlarged scale plan view of an airway tissue bioreactor which forms part of the system;

FIG. 2A is a perspective view of the airway tissue bioreactor shown in FIG. 1B;

FIG. 2B is a perspective view of the airway tissue bioreactor of FIG. 2A integrated with a micro-optical fiber based imaging module;

FIG. 3 is a series of schematic overviews of epithelium removal and cell implantation in accordance with an embodiment of the present invention, illustrating epithelium clearance (FIG. 3A); hydrogel-assisted cell seeding (FIG. 3B); and replacement of all or part of a patient's damaged airway or trachea with a bioengineered airway or trachea (FIG. 3C);

FIG. 4A is a perspective view illustrating apparatus, including a vibration stage, for mechanically vibrating a trachea using a custom-built electromagnetic shaker after cell lysis by detergent solution;

FIG. 4B is a plan view showing a trachea bioreactor on the vibration stage of FIG. 4A in accordance with an embodiment of the present invention;

FIG. 4C is a schematic of a component of the custom-built electromagnetic shaker of FIGS. 4A and 4B;

FIG. 4D is a graph of accelerative waves generated at frequency 20 Hz by a computer, in accordance with an embodiment of the present invention;

FIG. 4E is a graph illustrating output acceleration vs. time for the electromagnetic shaker of FIGS. 4A and 4B;

FIG. 4F is a graph illustrating displacement of the vibration stage of FIG. 4B as monitored by a high-speed camera and processed with the ImageJ;

FIG. 5 is a schematic showing an imaging probe being used for visual inspection of an in vitro-cultured airway tissue, in accordance with an embodiment of the present invention;

FIG. 6A is a photograph (i) and a schematic (ii) illustrating an optical fiber (diameter: 500 μm) used for both bright-field and fluorescence imaging of a rat tracheal lumen, in accordance with an embodiment of the present invention;

FIG. 6B is a photograph showing an imaging probe being used for visual inspection of an in vitro-cultured airway tissue, in accordance with an embodiment of the present invention;

FIG. 6C is bright-field (i) and fluorescence (ii) images of an airway interior before CFSE-labeling of the epithelium, in accordance with an embodiment of the present invention;

FIG. 6D is fluorescence images of native (i) and de-epithelialized (ii; De-epi) airway lumen that were labeled with CFSE, in accordance with an embodiment of the present invention;

FIG. 6E is a series of images showing in situ fluorescently labeled cells deposited onto an inner lumen of airway tissue, in accordance with an embodiment of the present invention;

FIG. 7A is a schematic illustration showing mucociliary clearance monitoring via in situ particle tracking of fluorescent microparticles to assess establishment of mucociliary function;

FIG. 7B is an image showing mucociliary clearance monitoring via in situ particle tracking, wherein fluid movement is determined by tracking 1-μm microparticles;

FIG. 7C is a series of images showing mucociliary clearance monitoring via in situ particle tracking of multiple microparticles with reduced magnification;

FIGS. 8A-8B are schematic illustrations of bioartificial mucus with a tunable range of viscoelasticity, in accordance with an embodiment of the present invention;

FIG. 9A is a schematic diagram illustrating instillation of mucus or synthetic mucus mimics to generate a thick, viscous mucus film onto the inner lumen of ex vivo airway, in accordance with an embodiment of the present invention;

FIG. 9B is a series of photographs demonstrating an even coating of fluorescein-dyed bioartificial mucus onto the inner surface of human-sized secondary bronchus, in accordance with an embodiment of the present invention;

FIG. 9C is a cross-sectional view of a tubular channel made of gelatin onto which the indocyanine green (ICG)-labeled collagen hydrogel is homogenously coated, in accordance with an embodiment of the present invention;

FIG. 10A is a schematic representation illustrating a pressure and flow control system capable of generating an airflow that mimics coughing and natural breathing within the in vitro airway tissue, in accordance with an embodiment of the present invention;

FIG. 10B is a schematic representation illustrating a pressure and flow control system capable of generating an airflow that mimics coughing and natural breathing within the in vitro airway tissue, in accordance with an embodiment of the present invention;

FIGS. 11A-11B are schematic representations illustrating delivery of therapeutic agents (e.g., drug molecules or gene editing machinery) into the in vitro-cultured airway tissue via liquid instillation (FIG. 11A) and aerosol inhalation (FIG. 11B);

FIGS. 12A-12E are three-dimensional (3D) computer drawings of the airway bioreactor, showing a top plan view (FIG. 12A), a side elevational view (FIG. 12B), and various dimensional drawings (FIGS. 12C-12E);

FIG. 13 is a schematic diagram showing media flow directions in the airway lumen and in the bioreactor chamber, wherein Flow A passes through the airway lumen via inlet (1), and Flow B passes in the bioreactor chamber and supply the exterior surface of the airway via inlet (2), wherein such imaging tools can be inserted through inlet (1) to visualize the airway lumen;

FIGS. 14A-14B are schematics showing an experimental setup used for long-term in vitro culture of the cells in the airway lumen, in accordance with an embodiment of the present invention, wherein Pump #1 (FIG. 14A) supplies the culture medium through airway lumen and pump #2 (FIG. 14A) supplies the culture medium to the exterior surface of airway in the chamber, with the arrows showing flow directions (FIG. 14A); and

FIG. 15 is a table listing details of component parts that can be used as attachments for a bioreactor in accordance with embodiments of the present invention.

### DETAILED DESCRIPTION OF THE EXEMPLARY EMBODIMENTS

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign thereto.

Further, it should be noted that, as recited herein, the singular forms “a”, “an”, “the”, and “one” include the plural referents unless otherwise stated. Additionally, the terms “comprises” and “comprising” when used herein specify that certain features are present in that embodiment, however, this phrase should not be interpreted to preclude the presence or addition of additional steps, operations, features, components, and/or groups thereof.

All examples and conditional language recited herein are intended for pedagogical purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

The present invention involves an integrated airway bioreactor system and methods that can be used for (i) application of user-defined mechanical and biochemical treatments to in vitro-cultured ex vivo airway tissue obtained from human lung; (ii) real-time monitoring of the interior or exterior surfaces of the ex vivo airway tissue at the cellular level; (iii) controllable removal of endogenous cells and subsequent uniform distribution of newly implanted cells; (iv) long-term in vitro cultivation and manipulation of ex vivo airway tissue; (v) in vitro recapitulation of impaired mucociliary flow; (vi) imaging-assisted assessment of the impact of mechanical and biochemical treatments on mucociliary function reestablishment; and (vii) in vitro investigation of the effects of coughing on clearance of mucus fluid and subsequent reestablishment of mucociliary flow. Similarly, isolated airway tissues obtained from large animals (e.g., swine) can be also used in the bioreactor.

The airway tissue bioreactor system can include a custom-built tissue culture chamber, in which isolated airway tissue can be placed and cultivated during in vitro tissue manipulation and investigation. The tissue culture chamber is connected to a computerized ventilator and a perfusion pump to provide a near-native environment to the cultured tissue. A sample perfusion pump that can be used with the present invention is a model: (L/S® standard digital pump system, Cole-Parmer). The computerized ventilator represents a coughing system in the context of the present invention. Additionally, using the same coughing system, controlled airflow can be introduced in and out of the airway tissue within the bioreactor, accurately mimicking the physiological conditions of natural breathing.

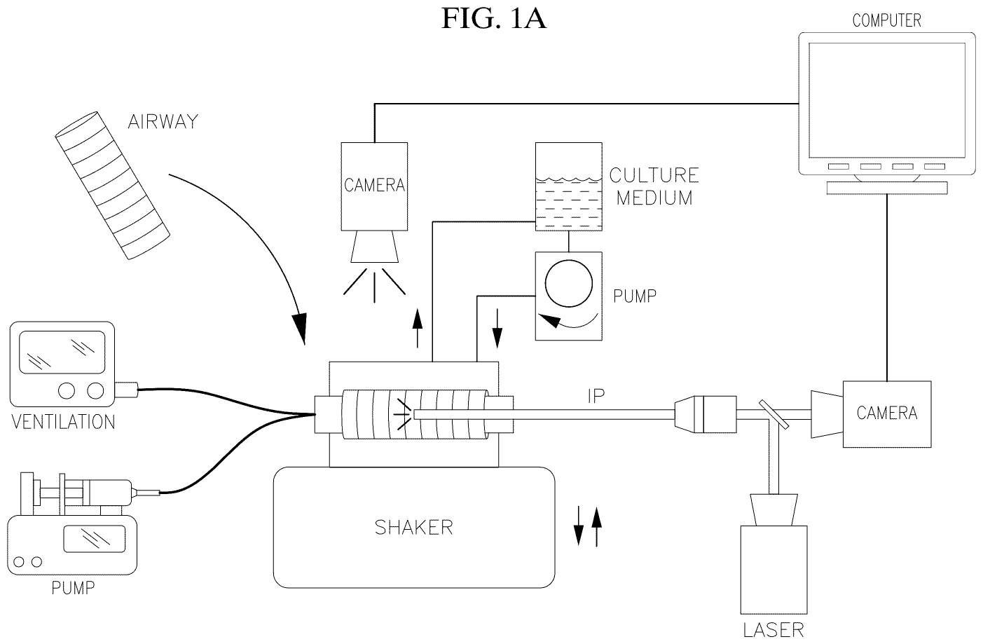

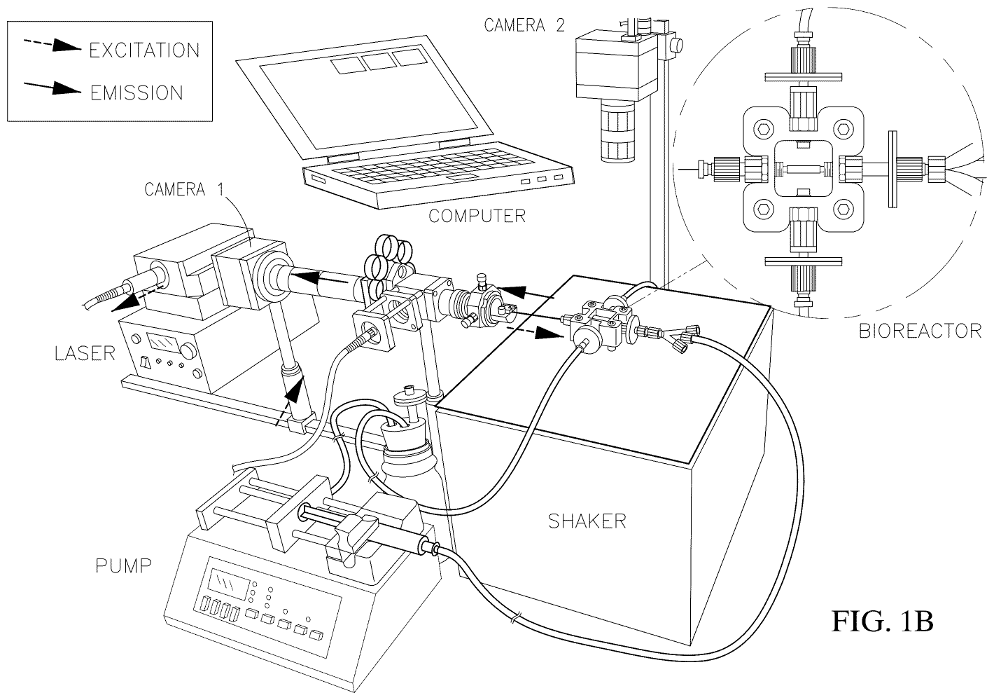

The tissue chamber can be placed onto a shaker to provide oscillatory mechanical force to the tissue and connected to a pump to introduce biochemical reagents or cells to the airway tissue. To promote long-term viability of the thick tissue being cultured, artificial hemoglobin or perfluorocarbon oxygen carriers can be added to the culture medium (FIG. 1). These elements can be used to enhance oxygen solubility and efficient oxygen transport within the culture medium. Both components are in liquid states and can be directly added to the culture medium

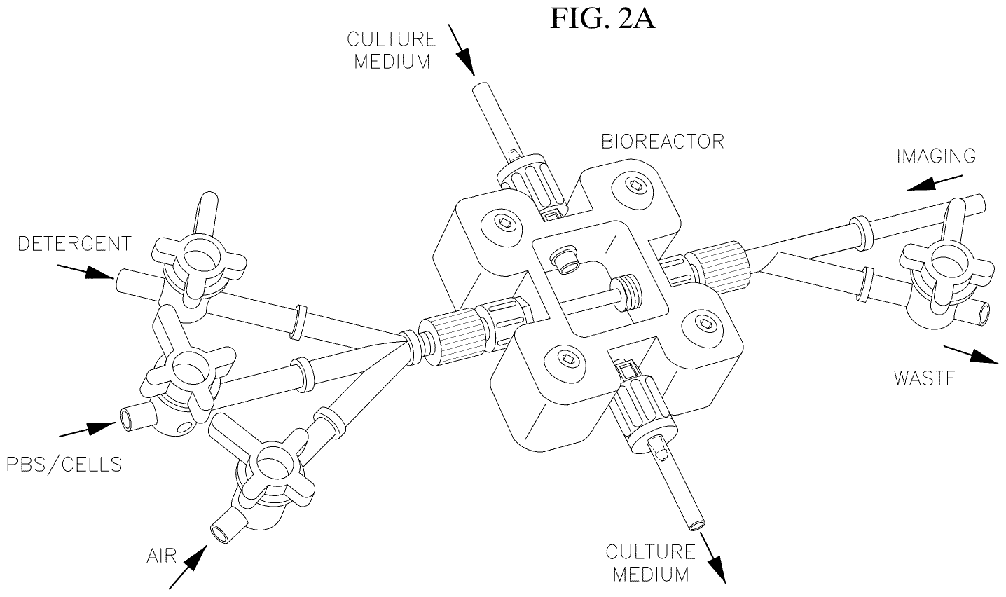

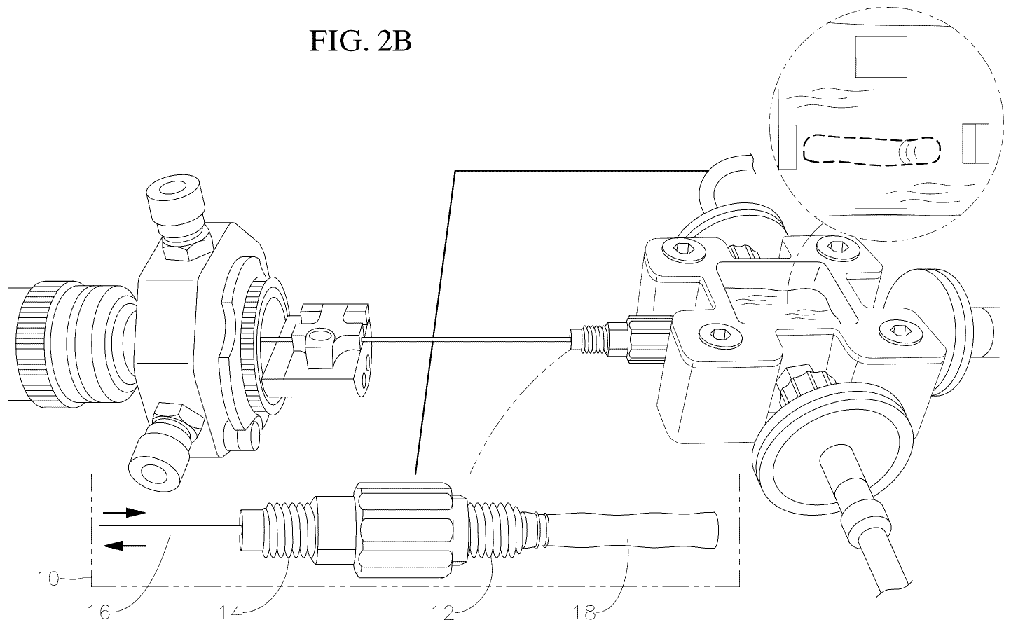

An optical fiber-based imaging modality is coupled to the bioreactor system to allow real-time in situ monitoring of the luminal surface of the airway tissue at the cellular (e.g., single-cell) level, thereby enabling visually guided tight regulation and confirmation of tissue manipulation (FIG. 2). Pictured in FIG. 2A is a waste outlet that can be used to remove the biological agents (e.g., detergents, enzymes, etc.), phosphate-buffered saline solution (PBS), and used culture medium from the airway lumen.

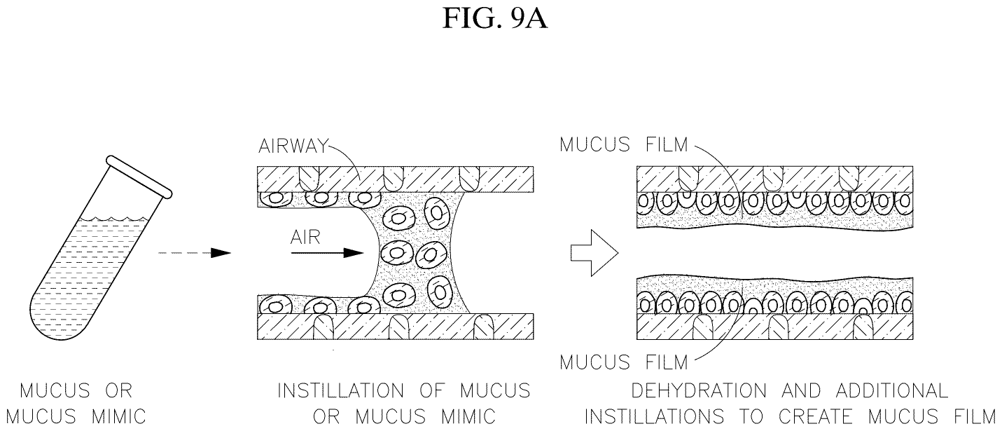

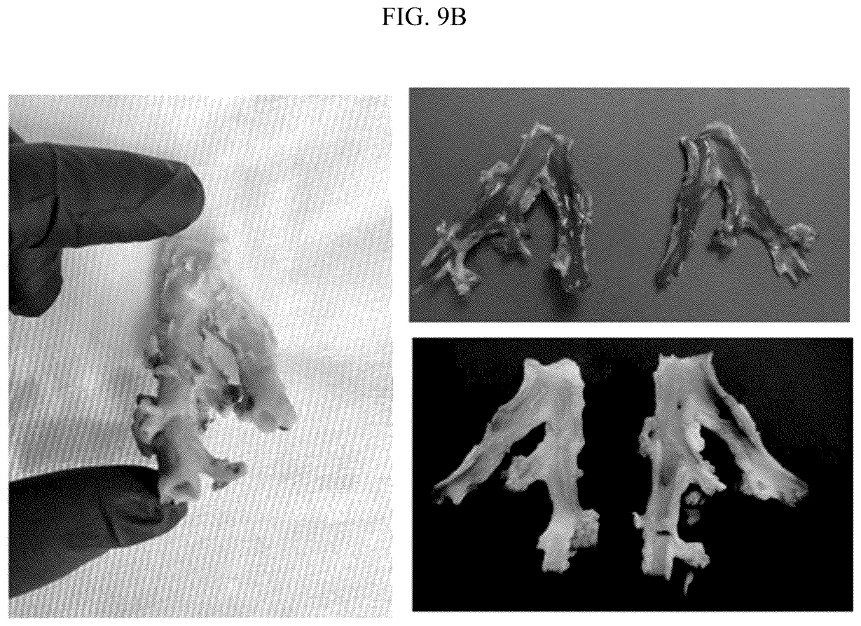

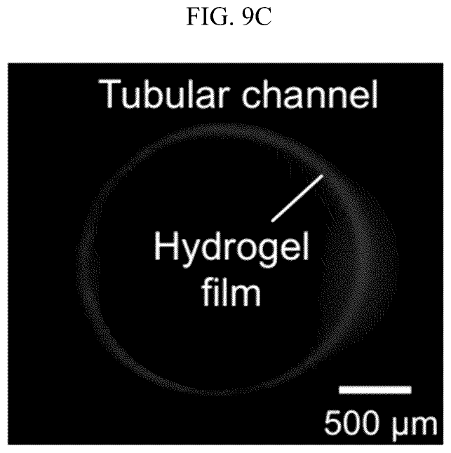

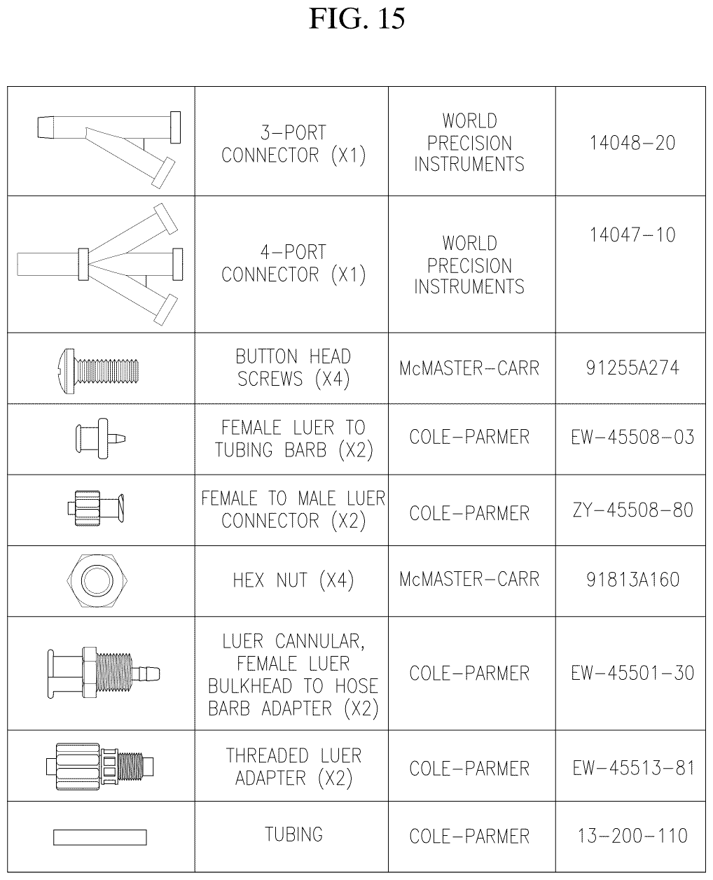

The connections from the bioreactor to a micro-optical fiber-based imaging module 10 are shown in FIG. 2B: a Luer cannula 12 is shown connected to a threaded Luer adaptor 14 and the imaging probe 16 on one side and to the airway on the other side with silk thread 18 (See FIG. 15, items 5 and 7, and their usage in FIG. 9B and FIGS. 1A-1B). The silk thread is used to secure the airway tissue on the Luer cannula to prevent any leakage of regents/solution introduced into the airway lumen to the bioreactor chamber (airway exterior surface).

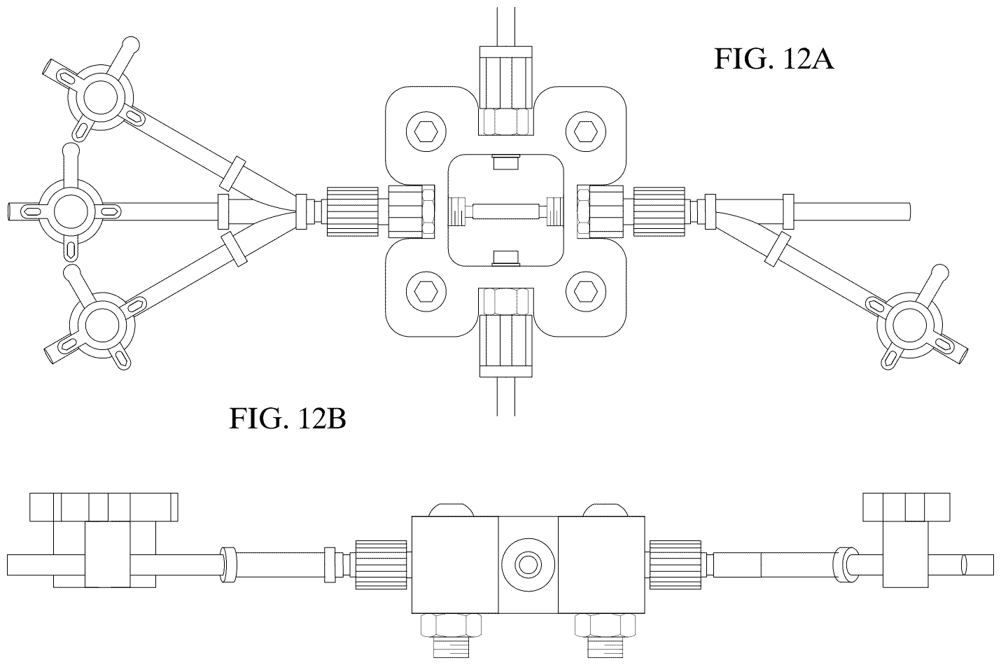

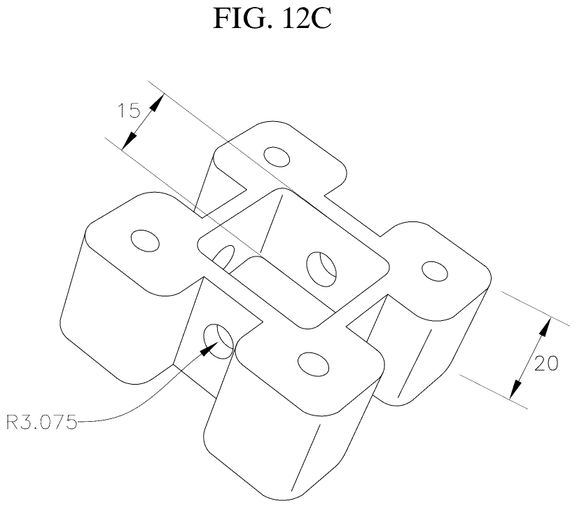

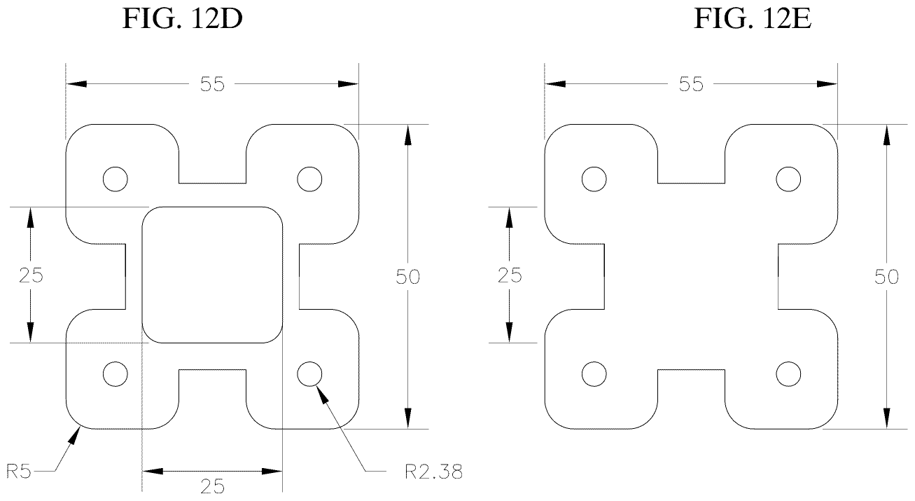

To create the tissue culture chamber a computer-aided design (CAD) model of the chamber can be generated using CAD generator software, such as SolidWorks and Fusion-360 (FIGS. 12A-12E). The CAD model includes the dimensions of the tissue culture chamber with inlets, outlets and connectors (FIGS. 12C-12E). Next, the generated CAD model is exported to a computer numerical control (CNC) controller software. The CNC machine cuts, for instance, the polytetrafluoroethylene (PTFE) plastic based on the CAD model. In the embodiment of FIGS. 12A-12E, a transparent acrylic plastic sheet is cut and attached to the top of the main chamber using screws (FIG. 12E). Unit=mm; R=radius. Furthermore, in addition to PTFE, other plastic materials, such as ultra-high molecular weight polyethylene (UHMWPE) and polyetherimide (PEI) can be used to construct the culture chamber. Plastic tubes are attached to the bioreactor chamber through various Luer connectors including male, female, 3-port, 4-port Luer connecters, which are integrated to the bioreactor chamber. A list of the tissue chamber attachments is presented in FIG. 15. The chamber connections enable multiple functions: instilling various reagents (detergent, enzymes, etc.) and washing solution, the delivery and circulation of culture medium in the chamber. Additionally, the thin imaging probe can be introduced through the Luer connector into the airway lumen for, for example, visualizing the airway luminal surface during removal of epithelial cell and introduction of new cell populations (see Examples 1 and 2 below).

The Luer connectors, such as Luer cannulas 12, can be fixed to both ends of the tissue culture chamber to facilitate the insertion of the airway tissues into the chamber. The two ends of the airway tissues can be secured to the Luer cannulas 12 with sutures (FIG. 2B). In addition, the chamber's design can be customized to accommodate the specific dimensions and characteristics of the airway tissue segments from human or animal sources. Moreover, the chamber's design facilitates the precise control and circulation of fluids, including culture medium and reagents, in a controlled and monitored environment for the airway tissue segment. By using a vacuum pump or air compressor, controlled pressure can be applied within the airway tissue, replicating the mechanical forces present in natural breathing and coughing. Meanwhile, the precise regulation of air flow during inhalation and exhalation within the airway allows mimicking the airflow dynamics of the respiratory system. In particular, the utilization of a thin film-based method (coating method) allows for the manipulation of airway tissues and cells (including de-epithelialization, new cell population, therapeutic delivery, etc.) while maintaining ventilation in a manner that closely resembles the natural airway. Furthermore, the chamber's design accommodates optical fiber-based imaging modules, enabling the visualization and monitoring of airway lumen and studying its behavior and response to various treatments.

The bioreactor chamber is designed and constructed in a way that the luminal surface of the airway can be treated using different solutions (e.g., decellularization solution, washing solution, cells, culture medium, etc.) while the entire exterior surface of the airway tissue is submerged in a cell culture medium to maintain the viability of the airway tissue during experiments.

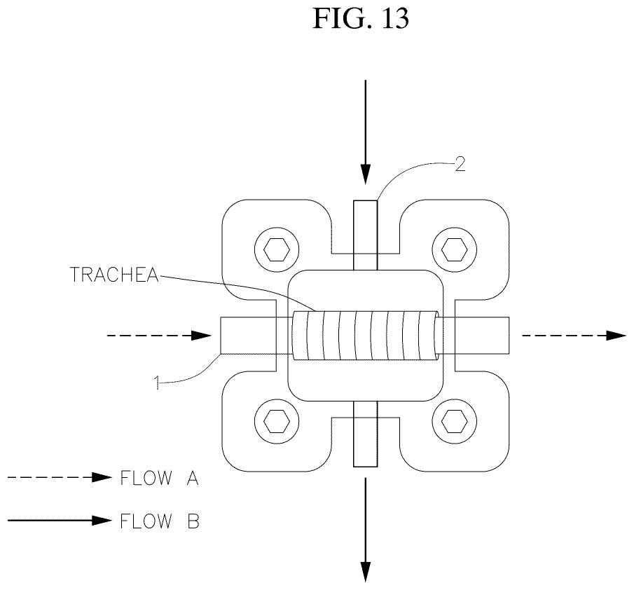

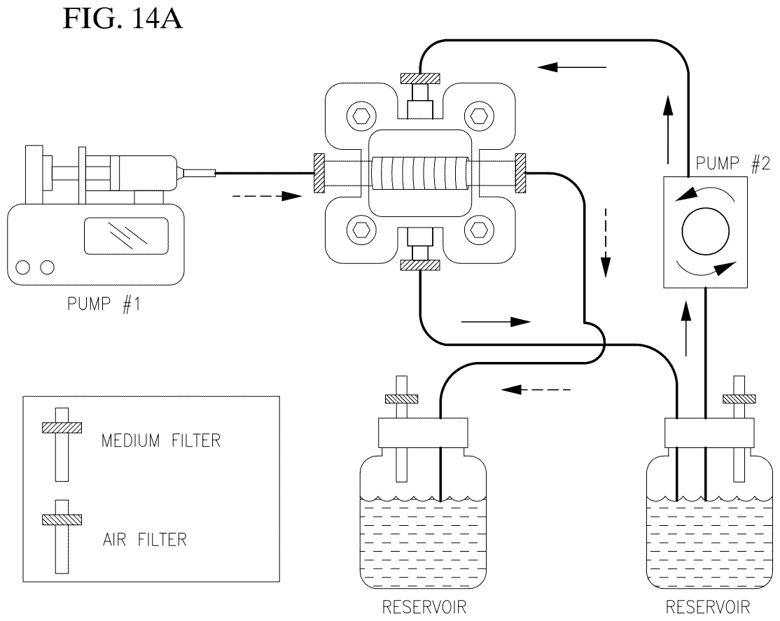

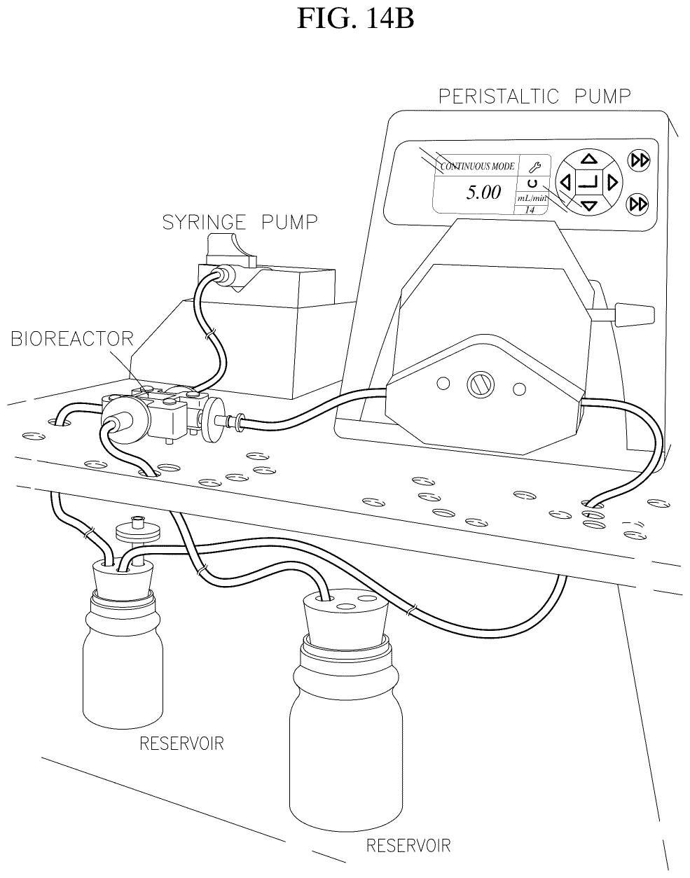

Furthermore, this design enables introduction of the imaging fiber into the airway lumen for visualization purposes, while the exterior surface of the airway tissue is supplemented with the culture medium (FIG. 13 and FIGS. 14A-14B). This design feature allows in situ optical fiber-based imaging of the airway tissue throughout long-term cell culture, without removing the airway from the bioreactor.

By way of example, the culture medium connected to the culture chamber can contain Dulbecco's modified Eagle's medium (DMEM; Gibco™, Cat. No. 11965118, Thermo Fisher Scientific), recombinant human FGF-basic (Cat. No. 100-18B, PeproTech), fetal bovine serum (FBS; Gibco™, Cat. No. 10082147, Thermo Fisher Scientific), and antibiotic-antimycotic (Cat. No. 15240062, Thermo Fisher Scientific). Hemoglobin or perfluorocarbon (PFC)-based oxygen carriers can be added to the culture medium to enhance oxygen solubility and efficient oxygen transport within the medium. This complete culture medium is used for supporting the cells cultured onto the airway lumen and the exterior surface of the airway tissue.

The culture medium circulates in and out of the bioreactor through different inlets and outlets. During the cell culture, the culture medium inside the airway lumen is supplemented with the medium using a programmable syringe pump (e.g., flow rate: 5 mL/h; volume: 10 mL; AL-4000, World Precision Instruments) (FIG. 13, Flow A, inlet (1)). Simultaneously, the culture medium within the tissue culture chamber that supports exterior airway tissue is continuously replaced by circulating the medium between a reservoir and the chamber using a peristaltic pump (flow rate: 5 mL/min; L/S® standard digital pump system, Cole-Parmer) (FIG. 13, Flow B, inlet (2)). For more details, please also refer to FIGS. 1A and 1B. The arrows in FIG. 1 entering and exiting the bioreactor chamber indicate circulation of the culture medium in the system.

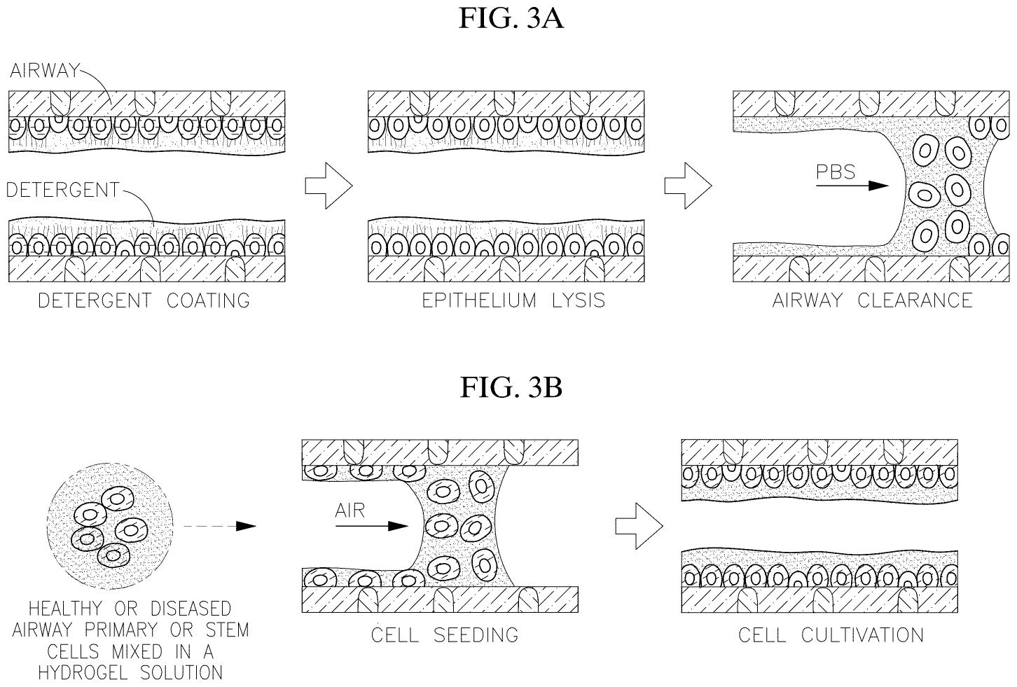

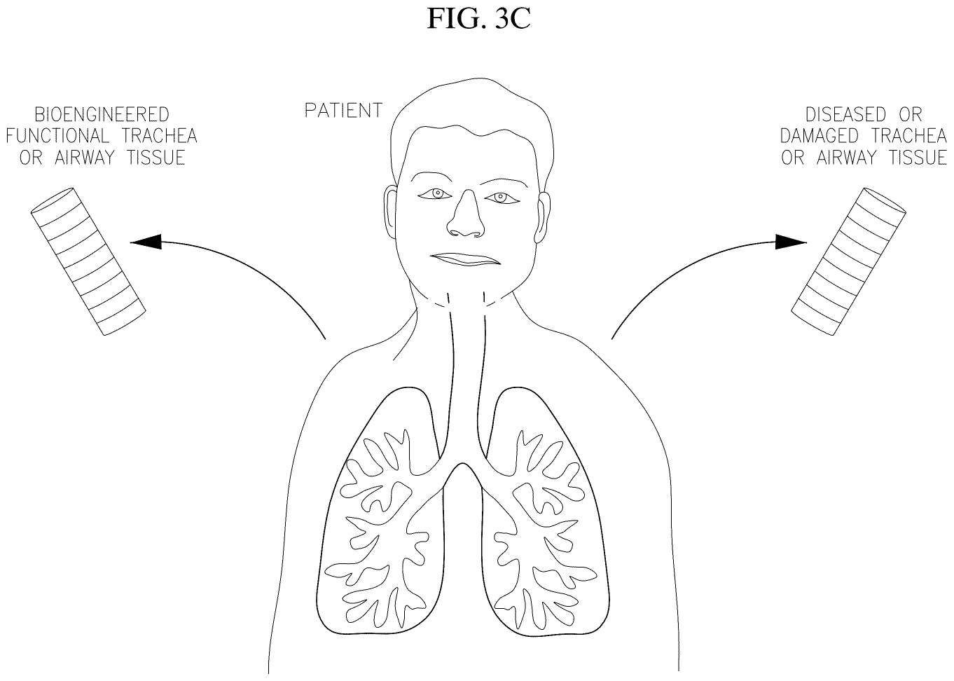

Using the bioreactor system, removal of the cellular components (i.e., de-cellularization) and repopulation of the tissue scaffolds with new airway cells (i.e., re-cellularization) can be achieved in a tightly regulated manner, thereby allowing in vitro generation of healthy or diseased airway tissue. Notably, by introducing de-cellularization reagents (e.g., detergents) with different strength, volume, and incubation time, the endogenous airway cells can be controllably removed while preserving the extracellular components (FIG. 3A). Homogenous deposition of exogenous cells can be achieved across the de-cellularized airway lumen by instilling the cells mixed in a hydrogel solution (e.g., collagen pre-gel) (FIG. 3B). The bioengineered airway tissue can be used to replace the entire or part of a patient's diseased or damaged airway tissue (FIG. 3C).

More specifically, FIGS. 3A and 3B are schematics showing the processes of epithelium removal and clearance, as well as cell seeding in the airway bioreactor, respectively. In the present invention, sodium dodecyl sulfate (SDS, concentration: 2% and 4%) can be used to lyse the airway epithelial cells. Subsequently, the mechanical vibration with PBS solution washing can be used to remove the lysed epithelium and to clear the airway. CF SE-labeled mesenchymal stem cells (MSCs) may be used as a cell model to seed the de-epithelialized airway. The cells were first labeled with CF SE, then suspended in collagen hydrogel, and then instilled onto the airway luminal surface. Other cell types, such as airway primary cells and stem cells, can be used as the cell sources. Alternatively, the diseased airway cells can be used to create various airway disease models in the bioreactor.

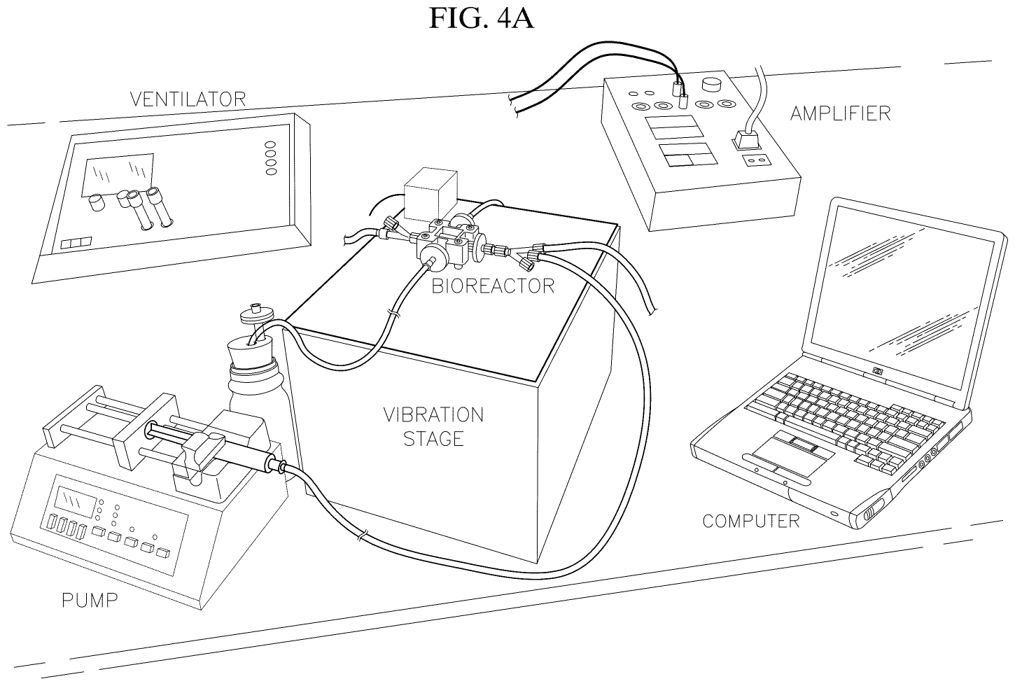

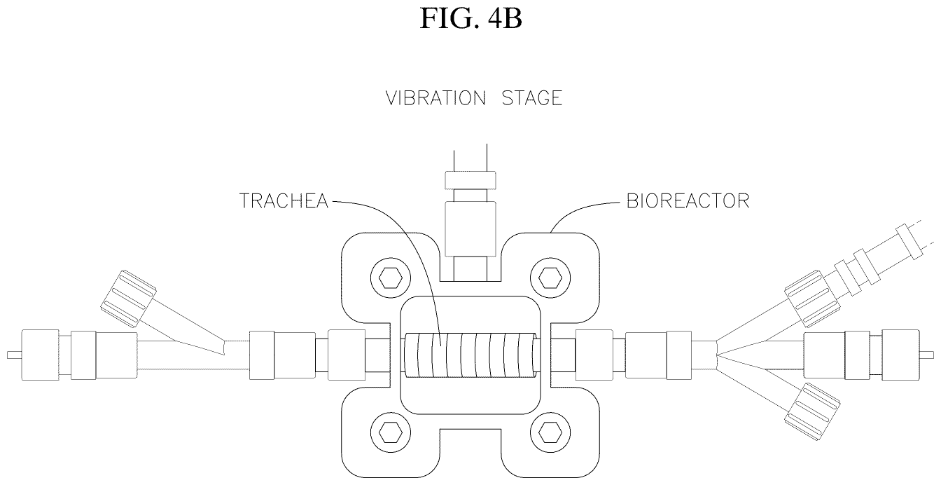

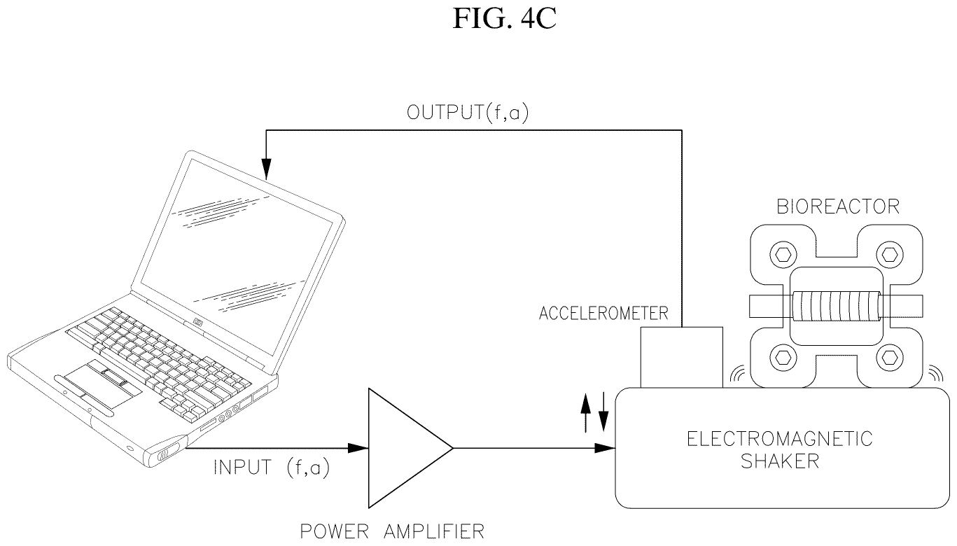

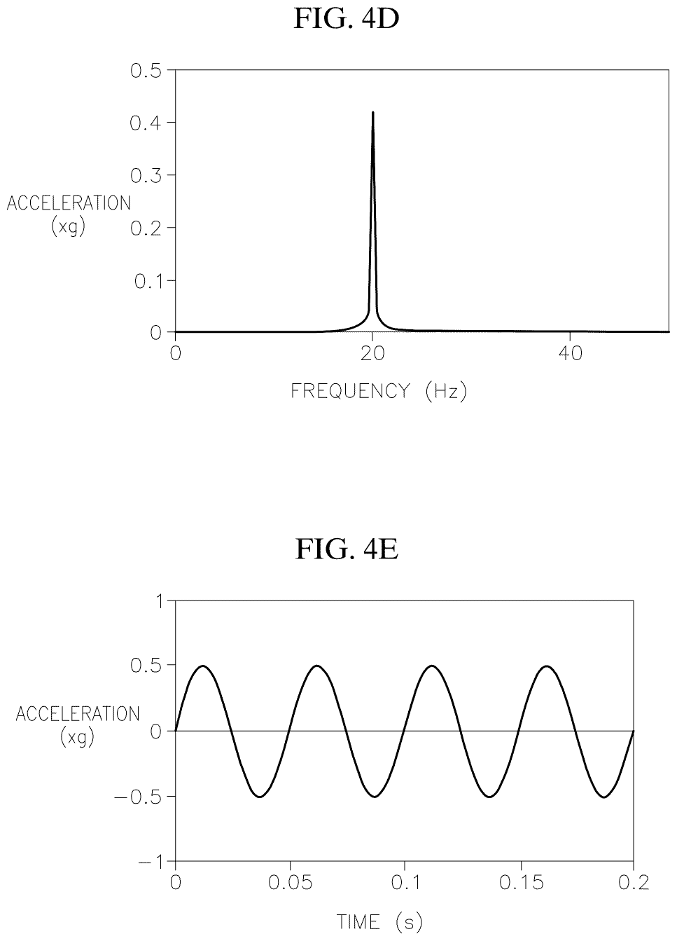

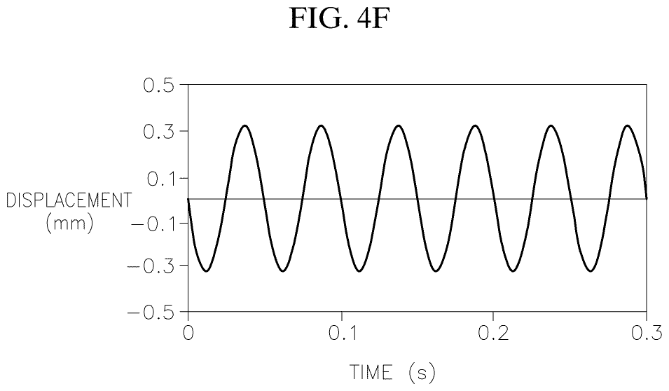

A computer-controlled shaker can generate user-defined mechanical vibration forces to the airway tissue located inside the cultivation chamber. By modulating frequency, amplitude, and waveform shape of the vibration, the magnitude of the force can be adjusted (FIG. 4). FIG. 4 pertains to mechanical vibration for clearance of detergent-disrupted epithelial cells. We used a subwoofer plate amplifier (SPA250DSP, Dayton Audio) in the present invention. The shaker can include an amplifier. For instance, it can be a subwoofer plate amplifier, which is a specialized electronic component designed to power and control a subwoofer speaker in an audio system. Here, the plate amplifier is shown which provides required power to the speaker cone and controls the vibration of the speaker cone to the desired amplitude. Also shown, in FIG. 4C, is an accelerometer (e.g., Model: IIS3DWBTR, STMicroelectronics) which measures the frequency and amplitude of the oscillation forces generated by the shaker and applied to the airway tissue within the bioreactor. The accelerometer verifies the input conditions generated by the computer and transmitted to the shaker are consistent with the shaker output. It can also accurately measure the same frequency and amplitude, regardless of its positioning on the top of the shaker.

An alternative method to measure frequency and amplitude (or displacement) is to use an iPhone camera (slow-motion mode). To do this, videos can be recorded at 240 frames per second (fps) and stored in AVI file format which is a standard video file format compatible with ImageJ. To determine the displacement distance of the stage, a ruler is placed adjacent to the shaker as a reference object with known length. The video file is then imported to ImageJ as a sequence of image frames using “AVI reader” plugin. The displacement distance of the sample stage in the image sequence can be measured with respect to the reference ruler. To improve visibility of the stage movement, “enhance contrast” and “find edges” functions were used in ImageJ. To plot the displacement curves, a small region of the stage moving up and down may be cropped and extracted from each image frame. The cropped images are then stitched horizontally with the “grid/collection stitching” plugin as a single image file. A similar procedure can be done for different regions of the stage to generate a continuous waveform that represents displacement of the sample stage over time (FIG. 4F).

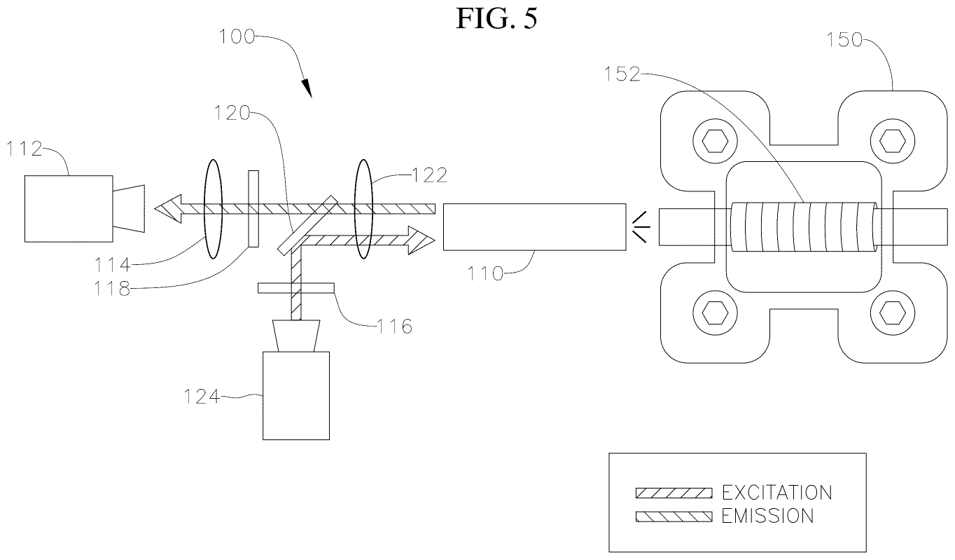

An optical fiber-based imaging probe (diameter: −500 μm) can be locally introduced into the airway tissues 152 to fluorescently visualize the airway lumen or cells in real time (FIG. 5). To visualize the inner lumen of the airway in either bright field or fluorescence, a micro-optical imaging module 100 was developed that can be integrated with the bioreactor 150. The imaging module includes an imaging probe 110 (GRIN lens; diameter: 500 μm; SELFOC®, NSG Group), a scientific camera 112 (PCO Panda 4.2), an achromatic doublet 114 (tube lens; AC254-150-A-ML, Thorlabs), two filter lenses 116, 118 (ET535/50m, Chroma®), a dualedge super-resolution dichroic mirror 120 (DI03-R488, Semrock), and a 20′ objective lens 122 (UCPLFLN20′, Olympus). For imaging the airway lumen, the distal end of the GRIN lens (imaging probe) is inserted into the Luer cannula that is attached to one end of the airway. Light signals emitted from the luminal surface of the airway are detected at the proximal end of the GRIN lens, which are then visualized via the camera. For fluorescent imaging, a laser light 124 (e.g., wavelength: 488 nm, power: 150 mW; MDL-D-488-150 mW, Opto Engine) is directed into the imaging probe to excite either the CFSE-labeled epithelium or the CFSE-labeled populated cells. Fluorescent light signals generated from the CF SE (emission wavelength: 515 nm) pass through the dichroic mirror and the bandpass filter lens, before being collected and imaged by the camera.

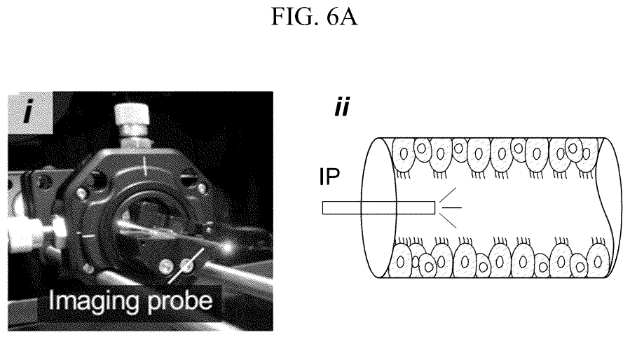

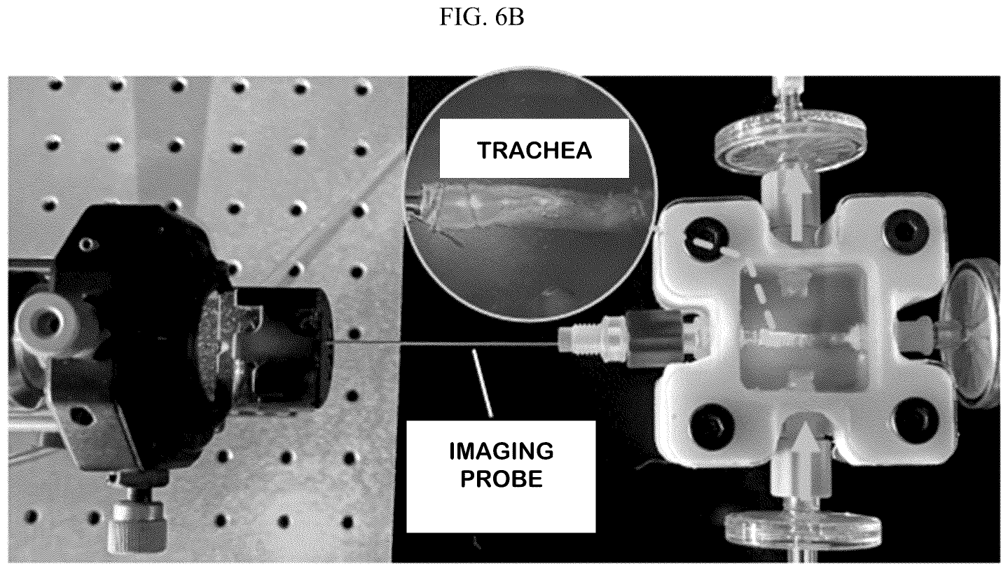

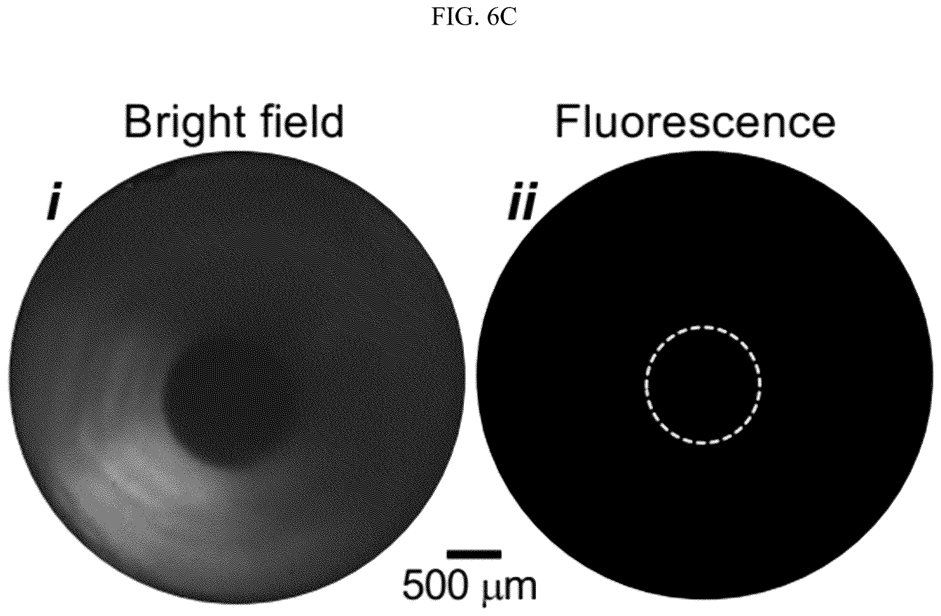

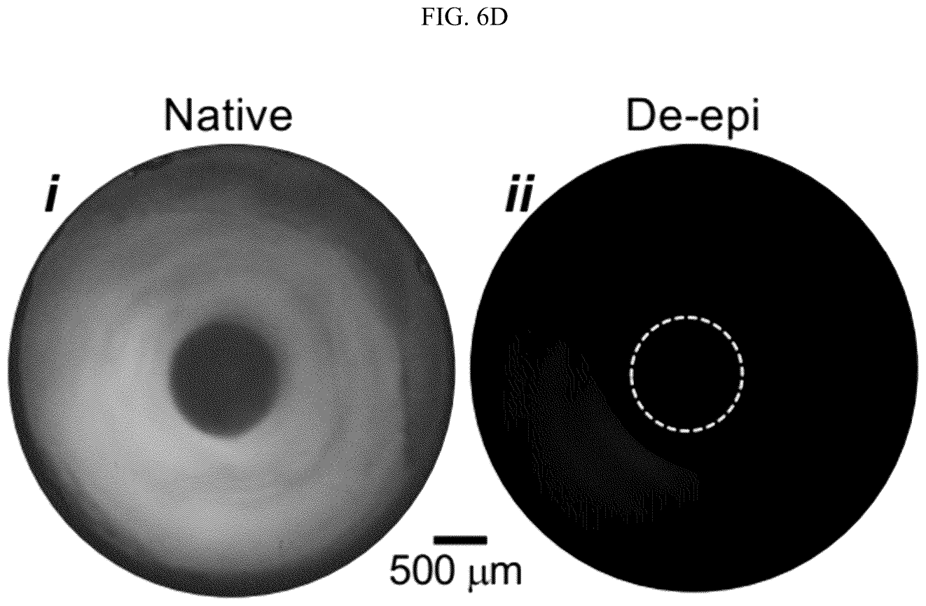

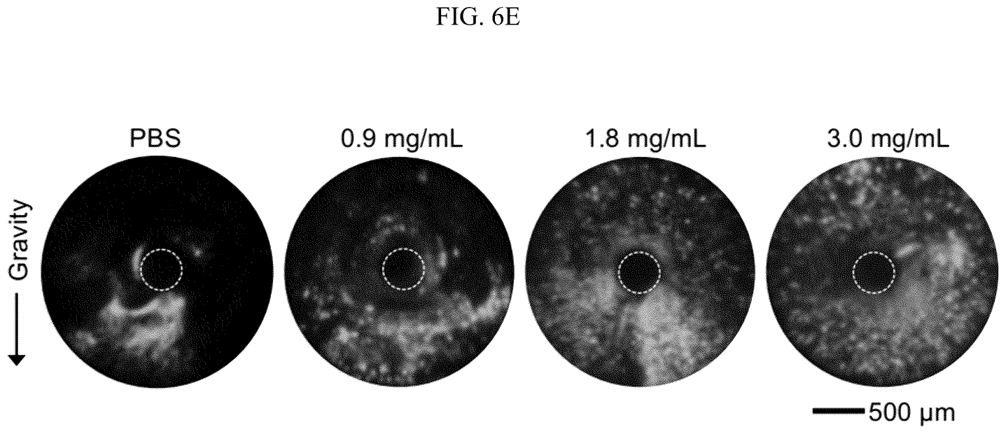

The top camera (Camera 2) visualizes the airway chamber (the exterior surface of the airway) from the top of the bioreactor (FIG. 1A). Internal imaging in the setups of FIG. 1B and FIG. 4B is effected with this camera. This camera is operable in both bright-field and fluorescent modes. The side camera (Camera 1) captures the images of the airway lumen visualized by the imaging probe (IP), and it is also operable in both bright-field and fluorescent modes (FIG. 1A). The images of FIGS. 6C-6E are taken with this camera. The laser light 124 enables visualization of the cells in the fluorescent mode. To capture fluorescent images, the laser light 124 (wavelength: 488 nm, power: 150 mW; MDL-D-488-150 mW, Opto Engine) is emitted into the imaging probe to excite the Carboxy Fluoroscein Succinimidyl Ester (CFSE)-labelled cells. Fluorescent light signals generated from the CFSE (emission wavelength: 515 nm) are then filtered by the dichroic mirror (model: DI03-R488, Semrock) and bandpass filter lens (ET535/50m, Chroma®), then collected and imaged via the camera. For example, FIG. 6D (i) shows the airway lumen with CFSE-labeled epithelium, and FIG. 6D (ii) shows the same lumen following removal of epithelial cells (i.e., De-epi). FIG. 6E shows the photos of the airway lumen populated with the CFSE-labeled cells.

The Dichroic mirror (DM) spectrally separates the light by transmitting and reflecting light based on the wavelength. In FIG. 5, the dichroic mirror (model: DI03-R488, Semrock) reflects the light generated by laser (wavelength=488 nm<495 nm), while allowing the transmission of the emission light (wavelength=515 nm>495 nm) from the cells to the camera (FIG. 5).

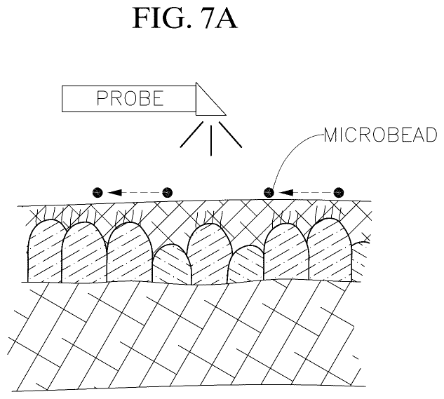

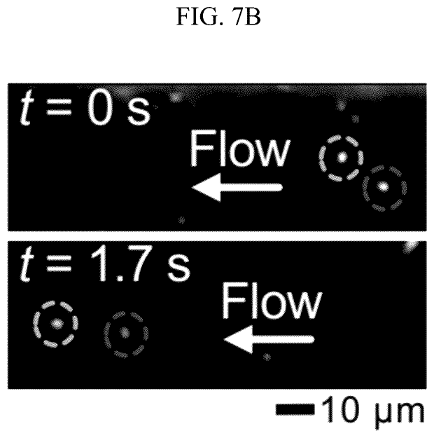

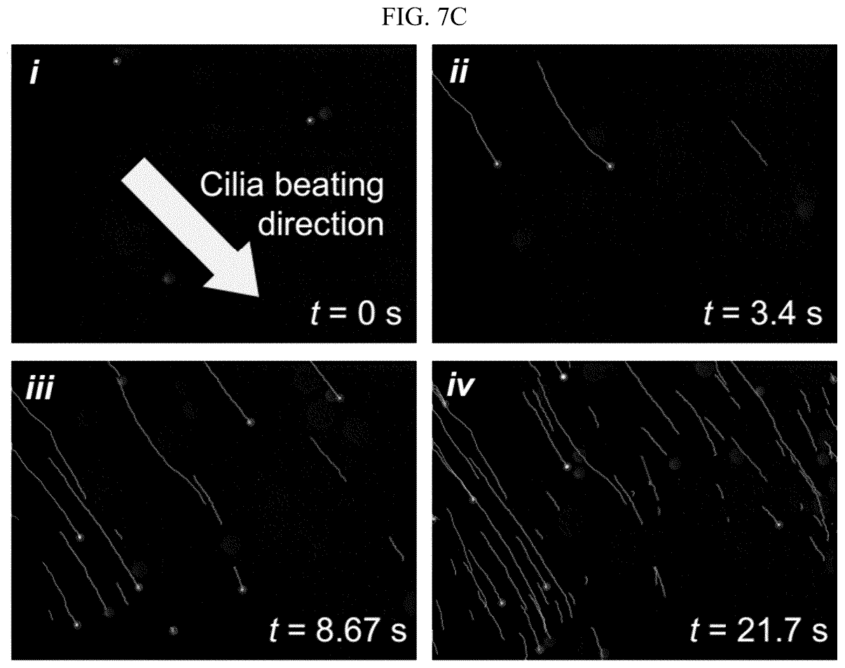

Notably, using this imaging approach, it is possible to non-destructively and rapidly confirm removal of the endogenous epithelial cells induced by the chemical and mechanical treatments without the need of conventional destructive and slow methods, such as biopsy, tissue sectioning, and immunostaining (FIGS. 6A-D). Further, using this imaging method, it is possible to monitor the distribution of the exogenous cells implanted onto the inner lumen of the ex vivo airway tissues (FIG. 6E). Using this imaging method, movement direction and speed of mucociliary flow over the surface of airway lumen can be accurately quantified by tracking microparticles (diameter: 1 μm) (FIG. 7B). Because diseased or injured airway tissues display substantially reduced mucociliary function, which can be confirmed by reduced particle movement speed, the microparticle tracking method can be used to diagnose lung diseases or lung tissue injury.

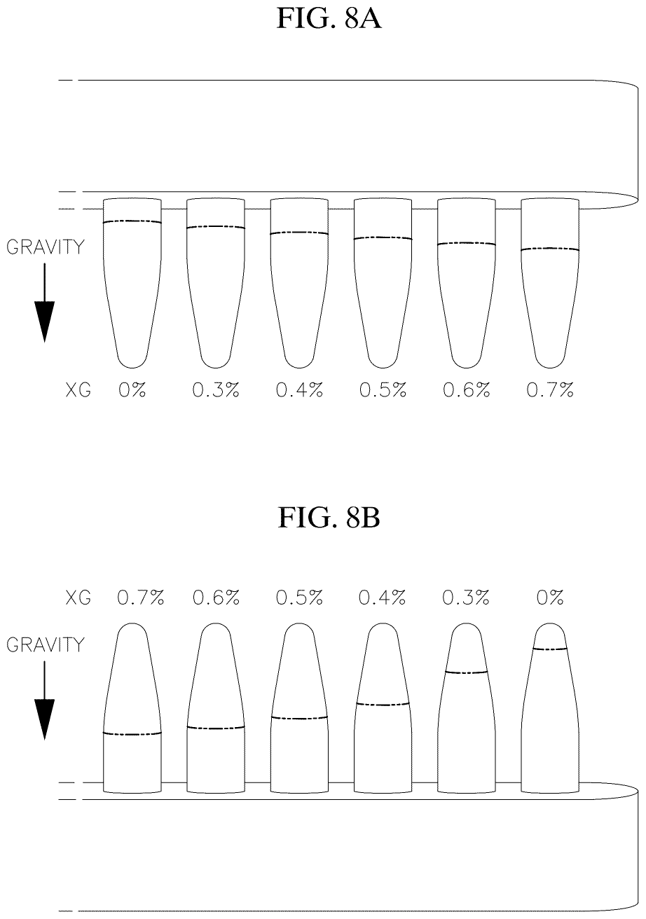

The airway bioreactor of the present invention can be used to create models of obstructive lung diseases associated with pathologic mucus buildup, such as cystic fibrosis (CF), primary ciliary dyskinesia, chronic bronchitis, and chronic obstructive pulmonary disease (COPD). Bioartificial mucus (BM) with tunable viscoelasticity (FIGS. 8A-8B) can be created by mixing biochemical materials, such as from mucin, low-molecular weight free DNA, salts and buffer (NaCl, KCl, Tris base, etc.), a lecithin source (e.g., egg yolk), a complete source of 20 amino acids (e.g., casein hydrolysate), and a low concentration (0-2%) of xanthan gum (XG) to provide the elastic component, with or without antibiotics. It has tunable viscoelastic properties provided by varying concentration of XG. FIGS. 8A and 8B depict a tilt test for bioartificial mucus containing XG 0-0.7%, showing a gradient of flow responses to force applied by gravity and agitation by tapping. BM can be instilled as a liquid or hydrogel into rodent to human-sized airways supported by the airway bioreactor, creating even, reproducible films that can be fluorescently labeled for further study (FIGS. 9A-9B). As illustrated in FIGS. 8A-8B, the composition and viscoelastic properties of this synthetic mucus can be tailored to replicate the characteristics of the mucus found in patients with cystic fibrosis (CF) and pulmonary ciliary dyskinesia (PCD), both of which involve impairment of the mucociliary function.

FIG. 9A illustrates a procedure for creating an airway disease model, such as for cystic fibrosis or COPD, characterized by impaired mucociliary function. The diseased airway model can be created by instilling synthetic mucus, which is delivered as a liquid plug to the airway to form a mucus layer on the luminal surface of the airway. To recapitulate the physical characteristics of the diseased airway, the deposited mucus can be allowed to dehydrate on the airway surface and additional mucus can be instilled to create a thicker mucus layer.

In FIG. 9B, photographs of the human-sized secondary bronchus are presented. Bioartificial mucus, labeled with fluorescein, is delivered as a liquid plug into the bronchus. To visualize the inner surface of bronchus and observe the mucus distribution across the airway lumen, the bronchus is bisected (right top photo). The left bottom photo shows fluorescent images of the bronchus, confirming the homogeneous distribution fluorescein-labeled mucus across the bronchus lumen.

FIG. 9C presents a photograph of a tubular channel made of gelatin hydrogel, designed to mimic the tubular geometry of an airway. To demonstrate the even distribution achieved with the hydrogel plug delivery method, first, the collagen hydrogel is labeled with indocyanine green (ICG) and then delivered as a plug into the gelatin channel. The bright line represents the collagen hydrogel layer, confirming the uniform distribution of hydrogel across the gelatin channel. The tubular channel depicted in FIG. 9C is made within the gelatin hydrogel to mimic tubular geometry of the airway. To create this tubular channel, gelatin powder (2.5 g, No. G2500, Sigma-Aldrich) is dissolved in 25 mL of deionized water at 70° C. for 10 min. The gelatin solution is then poured into a silicon container that contains a cylindrical metal rod (diameter: 2 mm) in the middle. The silicon container filled with molten gelatin is then placed in a refrigerator (temperature: 4° C.) for 30 min. After gelation, the metal is gently removed from the container by pulling it slowly, leaving a tubular structure within the gelatin. The gelatin tube is then removed from the silicon container and kept at 4° C. for re-use. The photograph in FIG. 9C shows the distribution of ICG-labeled collagen hydrogel across the gelatin tubular channel. Specifically, the ICG is initially mixed with the collagen pre-gel (in liquid state) and the pre-gel liquid plug is introduced into the tubular channel to deposit a homogenous pre-gel layer on the channel's surface (the ICG-labeled hydrogel indicated by a thin bright line). The pre-gel is subsequently allowed to cross-link within the channel. Such a procedure can be conducted to demonstrate homogenous distribution across the luminal surface of the airway mimic (gelatin channel) when collagen hydrogel is used as delivery vehicle.

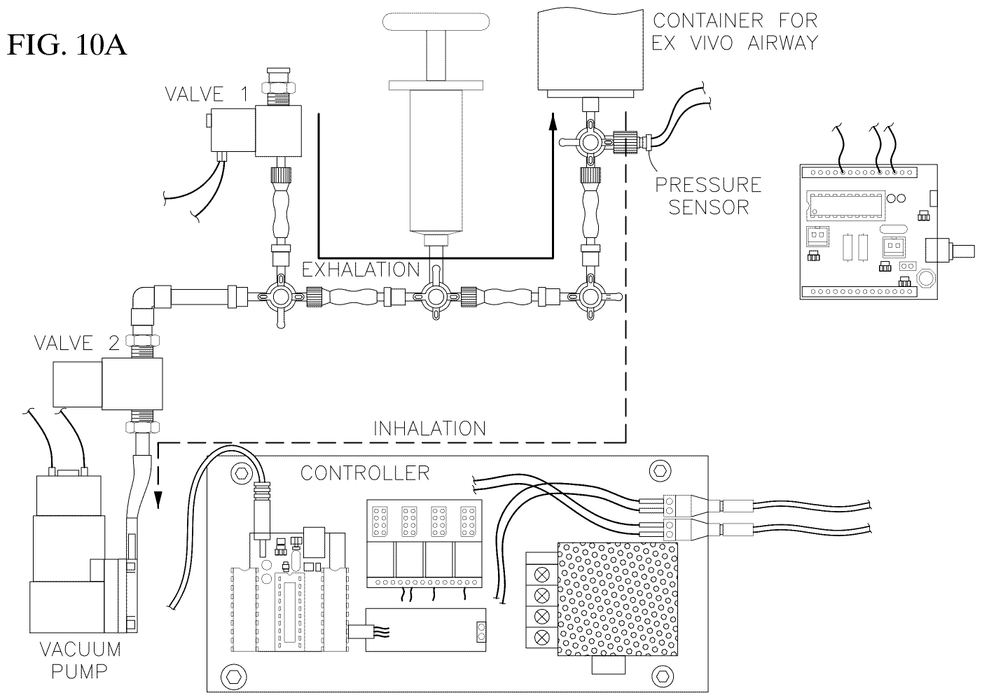

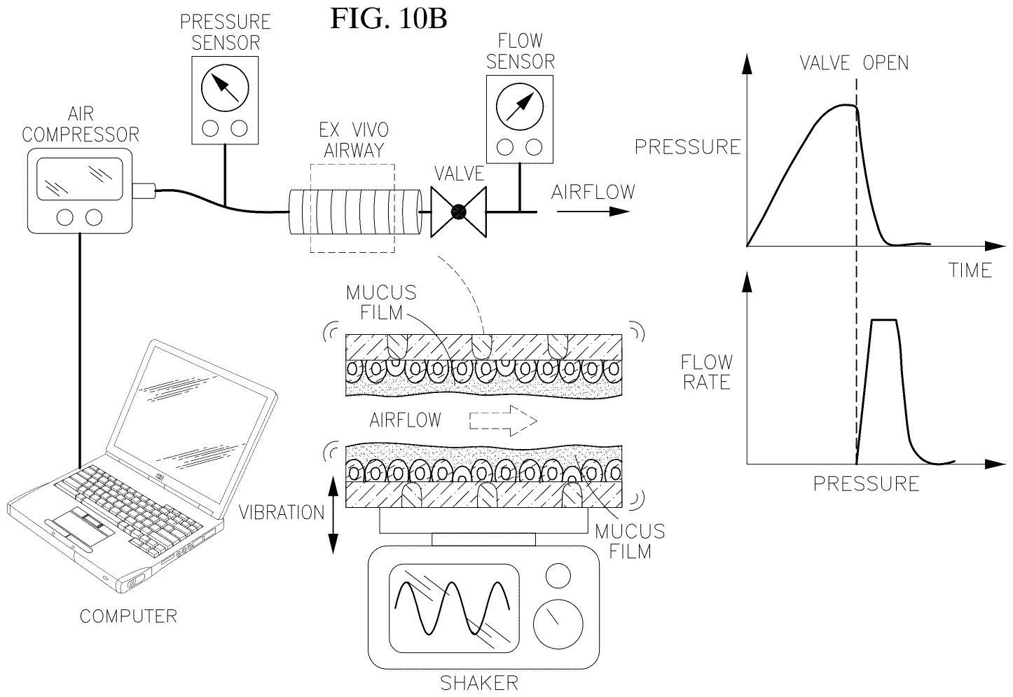

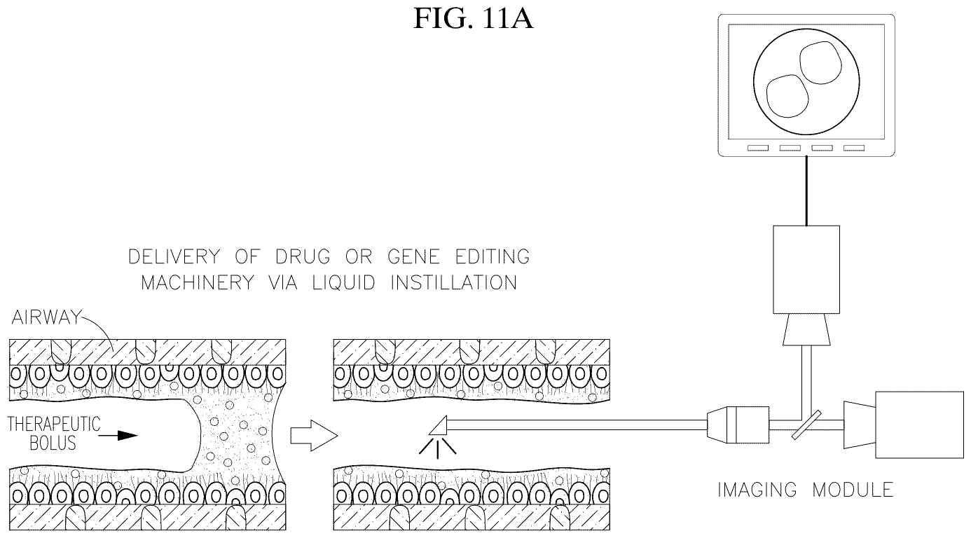

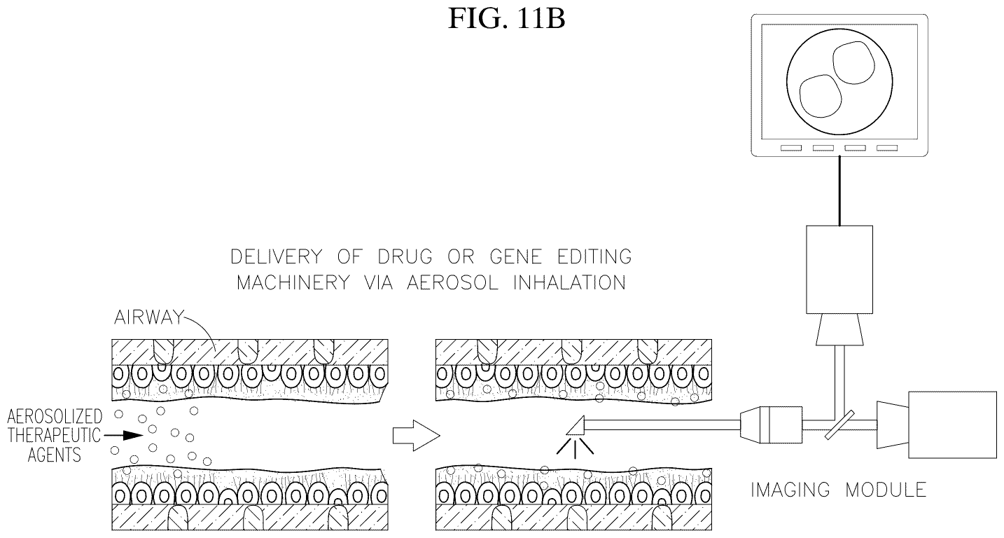

Studies supported by this model may include examination of mucociliary flow disruption by dehydrated mucus layers (FIG. 9A); mechanical and chemical disruption and clearance of mucus layers (FIG. 3A, FIGS. 4A-4F); and drug, virus, and nanoparticle transport through mucus to airway cells (FIG. 11). Beyond the standardized model created with bioartificial mucus, the airway bioreactor is also capable of supporting airways instilled with real patient mucus samples, as well as diseased airways derived from explanted human tissues. The airway bioreactor can be coupled with a cough generation system that can create a burst of airflow inside the airway tissue by modulating the air pressure and flow rate using a computer-controlled air compressor, pressure sensor, flow sensor, and valve (FIGS. 10A, 10B). Such a coughing system enables mimicking physiological microenvironments of the airway tissue during coughing or natural breathing. This integrated system can be used to investigate the impact of airflow on detachment and transport of thick mucus layers deposited onto airway lumen (FIGS. 10A, 10B). Specifically, clearance of mucus film via the airflow generated due to cough (FIGS. 10A, 10B) and mechanical vibration (FIGS. 4A-4F) can be studied using such a system. The in vitro-cultured airway tissue can be used to study the effectiveness of delivery of therapeutic agents, such as drug molecules, genes, or gene editing materials, via liquid instillation (FIG. 11A) or aerosol inhalation (FIG. 11B) followed by in situ optically based assessment (e.g., bioluminescent imaging) of the therapeutic outcomes (FIG. 5).

The setup in FIGS. 10A, 10B includes a system designed to simulate the airflow patterns during coughing or natural breathing within the ex vivo airway tissue in the container (e.g., airway bioreactor). Other breathing conditions that can be replicated are the breathing patterns of a healthy person or those of lung disease patients, such as patients with COPD, pulmonary fibrosis, or asthma. The breathing conditions can be modulated by generating negative pressure using a vacuum pump or positive pressure using an air compressor.

FIG. 10A illustrates the setup components for modulating and controlling of the airflow, including a pressure sensor (e.g., range: ±25 kPa; MPXV7002GC6U; NXP), a plastic container for ex vivo airway tissue, two normally closed valves (Valve 1 and Valve 2; Plum Garden; Voltage: 12 V), an airflow sensor that enables the measuring of volumetric airflow in the bioreactor container and tubing (flow range: ±750 cm3/min, HAFBLF0200CAAX5, Honeywell), a controller unit (DAQ, Arduino Uno), a computer and a vacuum pump (Model: D2028B Airpo; ABRA). The controller unit is a data acquisition (DAQ) device (Arduino Uno) that controls the vacuum pump and opening and closing of the Valve 1 and Valve 2 during inhalation and exhalation. Pressure data can be transferred to the computer via DAQ, then processed and plotted using custom-written MATLAB code (MathWorks). The pressure sensor measures the air pressure applied to the ex vivo airway within the container, generated by the vacuum pump. FIG. 10B is a schematic showing the pressure and flow control system. An air compressor can be alternatively used within the system to generate positive pressure on the ex vivo airway.

By using the in-situ imaging module integrated into the bioreactor, effects of the delivered therapeutics can be achieved via microscopic fluorescent imaging or bioluminescent imaging (FIGS. 11A-11B). FIG. 11A illustrates delivery of therapeutic agents, including drug molecules and genes, to the inner surface of the in vitro airway within the bioreactor, using either the liquid plug delivery method (as shown in FIG. 11A) or an aerosolized mixture (as shown in FIG. 11B). Following the deposition of the therapeutics onto the airway surface, whether through a liquid plug or an aerosolized mixture, the optical fiber-based imaging module (detailed in FIG. 5) can be employed to visualize and monitor the deposited therapeutics within the airway lumen.

### Example 1: De-Epithelialization of Luminal Surface

To remove the epithelial cells layer (i.e., epithelium) from the luminal surface of airway within the bioreactor, an aqueous solution of sodium dodecyl sulfate (SDS; concentrations: 2% or 4%) is instilled directly into the airway lumen via an inlet cannula connected to the bioreactor. In particular, a small volume (50 μL) of either 2% or 4% SDS solution can be infused through the airway lumen using a programmable syringe pump (e.g., flow rate: 6.3 mL/s; AL-4000, World Precision Instruments) to generate a thin film of the detergent solution on the luminal surface. To facilitate cell lysis exerted by detergent, the airway cam be incubated in the bioreactor for 20 min at 37° C. Subsequently, the bioreactor is mechanically vibrated using a custom-built shaker at 20 Hz of frequency while being washed with phosphate-buffered saline (PBS) solution three times (volume: 500 μL; flow rate: 10 mL/s).

The de-epithelialization technique of the present invention enables selective removal of the cellular components from the luminal surface of the cultured airway. This is achieved by the coating of the detergent onto the airway luminal surface using a plug delivery-based thin-film deposition method (FIG. 3A) followed by the mechanical vibration-assisted PBS washing (FIG. 3A and FIGS. 4A-4F).

A liquid coating technique enables precise deposition of a thin liquid layer onto the cultured airway tissues in the bioreactor. This technique allows targeted epithelium removal through topical deposition of a liquid solution containing decellularization reagents (e.g., detergent solution or enzyme) directly onto the airway lumen. By modulating the decellularization reaction time and detergent strength, the airway epithelial cells can be selectively disrupted and lysed.

The coating method developed in connection with the present invention provides a number of benefits including the small amounts of reagents required, usage of in situ tissue monitoring, and its translational potential.

The thin film method requires a small volume (less than 50 μL) of reagents, such as SDS detergent, for epithelium removal, while the flooding method would require much larger reagent volumes (at least −300 μL) to fill the airspace. Reduced amounts of reagents required can allow cost-effective airway tissue de-epithelization. Second, when the airway lumen is coated with detergent solution, visual inspection of the airway surface using a locally placed GRIN lens becomes easier than that of flooded airway following each step during the epithelium removal process (e.g., cell labeling, detergent film deposition, airway wash, and deepithelialization inspection). Inserting the imaging probe into liquid-filled airspace and imaging through the liquid medium are challenging within the flooded airway. Third, the thin film-based method could be more readily translated into clinics to treat patient's pathologic airway tissues when combined with innovative regenerative medicine technologies. For example, diseased or damaged epithelium could be removed from the airway lumen via the thin film approach and replaced with in vitro grown healthy primary airway epithelial cells or stem cells to regenerate the native epithelial layer within the patient's lung. On the other hand, the flooding method could cause substantial lung tissue damage as it does not permit localized and controllable reagent delivery within the geometrically complex respiratory tract.

Additionally, mechanical vibration-assisted washing also enables the use of smaller amounts of reagent with lower concentration for removing the cell components from the airway tissues. Typically, regents used to lyse the cells can damage the airway tissues, such as underlying basement membrane and extracellular matrix. Vibration-assisted airway washing can also reduce the detergent amount and required reaction time needed for lysing the epithelium, minimizing detergent-induced damage and detergent residue within the extracellular matrix.

### Example 2: Cell Replacement

Following de-epithelialization, the airway can be rinsed in 100′ Penicillin-Streptomycin for 1 min, 50′ culture medium with Penicillin-Streptomycin diluted by culture medium for 10 min, and Penicillin-Streptomycin for 1 min to decontaminate the airway grafts from the detergent and bacteria. Before cell seeding, the cells can be labeled with 100 μM Carboxyfluorescein succinimidyl ester (CFSE). After labeling, the labeled cells are redistributed in delivery liquid (PBS or collagen pre-gel). Immediately after redistribution, 10 μL of suspensions of the labeled cells are delivered into the airway lumen at a flow rate of 5 mL/min via tubing through a Luer connector. After cell injection, the culture medium within the airway lumen is supplemented with the fresh medium using a programmable syringe pump (flow rate: 1 mL/h; volume: 10 mL; AL-4000, World Precision Instruments) and the removed culture medium is collected in a waste reservoir. In the meantime, the culture medium within the tissue culture chamber of the bioreactor can replaced continuously by circulating the medium between a reservoir and the cell culture chamber using a peristaltic pump (e.g., flow rate: 5 mL/min; L/S® standard digital pump system, Cole-Parmer).

To homogenously repopulate the new cells onto the denuded (de-epithelialized) airway lumen, hydrogel can be used as the delivery vehicle for cells. To this end, the cells first suspended in the hydrogel solution, and then the hydrogel aliquots are delivered to the airway lumen.

One aspect of the cell population process involves the use of hydrogel to achieve spatially homogeneous cell seeding across the airway lumen. This method can provide a number of benefits compared with culture medium liquid-based deliver methods: 1) providing uniform cell distribution following cell seeding across the luminal surface of the airway, 2) creating an environment that allows survival and proliferation of the cells implanted onto the de epithelialized airway lumen.

### Example 3: Force Module

The force module can be a mechanical vibration platform used to generate user-defined mechanical vibration forces to the airway tissue within the bioreactor (FIGS. 4A-4F). In one embodiment, the module can be a custom-built shaker that is composed of a subwoofer speaker (RSS21OHO-4, Dayton Audio) and a subwoofer plate amplifier (SPA250DSP, Dayton Audio). A rigid acrylic plate (dimensions: 25 cm×30 cm×1.5 cm; McMaster-Carr) can be attached onto the diaphragm of the speaker (e.g., diameter: 21 cm).

The airway bioreactor is placed on the shaker plate during mechanical oscillation. The frequency and amplitude of the shaker can be controlled by feeding a computer-generated sinusoidal waveform to the shaker. The shaker's response is measured using an accelerometer (e.g., IIS3DWBTR, STMicroelectronics) or a high-frame-rate camera (e.g., 240 frames per second; iPhone 11 Pro). The force module is designed to apply vibration with customized frequency, amplitude, waveform shape for two purposes: 1) It can promote the detachment of the lysed cells during phosphate buffered saline (PBS) washing (FIG. 3A), or 2) It can enhance delivery of the therapeutic agents (drug molecules and genes) suspended in the liquid or aerosol mixture into the mucus and underlying airway extracellular matrix (ECM) and cells (FIG. 11).

### Example 4: Pressure Control Module

The pressure control module is responsible for regulating and measuring the air pressure that is applied to the airway tissue during the coughing process or user-defined airway tissue ventilation (FIG. 10). To modulate the pressure, either a vacuum pump (FIG. 10A) or air compressor (FIG. 10B) is employed to create specific pressure level within the system and tissue airway. Both the vacuum pump and air compressor can be controlled through a data acquisition (DAQ) device connected to the computer. Pressure generated by vacuum pump or air compressor is then measured by a pressure sensor (e.g., MPXV7025GC6U; NXP; pressure range±25 kPa) and a digital acquisition device (DAQ, Arduino Uno) connected to a computer. This pressure measurement enables the user to adjust the settings of the vacuum pump and air compressor to achieve the desired pressure in the airway lumen, allowing mimicking the airway pressure during the coughing or natural breathing.

### Example 5: Airflow Control Module

This module controls and measures the volumetric air flow (flow rate) during exhalation and inhalation processes (FIG. 10B). The flow control module uses a flow sensor flow range: ±750 cm³/min, HAFBLF0200CAAX5, Honeywell and a DAQ device (e.g., Arduino Uno) to measure the airflow during the inhalation and exhalation process. These data can be used to control the airflow generated by the vacuum pump and air compressor, accurately mimicking physiological conditions of airway during coughing and breathing.

This module enables long-term culturing of the airway stem cells (i.e., basal cells) in a ventilated environment. Following cell delivery, the cultured airway could be supplied with a gas flow (e.g., air) via the airflow system to the bioreactor to create air-liquid interface (ALI) within the airway and to provide in vivo-like flow shear stress to the seeded airway stem cells. This allows ALI environment to guide the differentiation of implanted basal cells into different epithelial cell types. The creation of ALI can be achieved by selectively controlling the infusion of culture medium and airflow through dedicated inlet ports. To create the ALI within the airway, a small volume (e.g., 50 μL) of culture medium can be periodically introduced through the inner space of the airway with liquid plug delivery instillation.

### Example 6: Liquid Flow Control Module

This module includes a programmable syringe pump (e.g., model: AL-4000, World Precision Instruments) and peristaltic pump (L/SR standard digital pump system, Cole-Parmer) to controllably instill various liquids, such as reagents (detergents and enzymes), washing solution, culture medium, pre-gel liquid, and bioartificial or patient-derived mucus into the airway within the bioreactor.

To support and culture newly seeded cells for long term, the airway-loaded bioreactor can be placed in a gas-controlled cell incubator (MCO-20AIC, Panasonic) at 37° C. and 5% CO2. During the cell culture, the culture medium within the inner space of the airway is replaced with the fresh medium once a day using a programmable syringe pump (flow rate: 5 mL h-1; volume: 10 mL; AL-4000, World Precision Instruments) and the removed culture medium can be collected in a waste reservoir. At the same time, the culture medium within the tissue culture chamber of the bioreactor is replaced continuously by circulating the medium between a reservoir and the chamber using a peristaltic pump (flow rate: 5 mL min-1; L/S® standard digital pump system, Cole-Parmer). The culture medium in the reservoir (total volume: 50 mL) may be replaced once in two days.

### Example 7: Custom-Built Shaker

To promote the detachment of epithelial cells from the airway lumen, a custom electromagnetic shaker, onto which an airway-loaded bioreactor can be placed and mechanically oscillated at a specified frequency and amplitude. The custom-built shaker is composed of a subwoofer speaker (RSS210H0-4, Dayton Audio) and a subwoofer plate amplifier (SPA250DSP, Dayton Audio). A rigid acrylic plate (dimensions: 25 cm×30 cm×1.5 cm; McMaster-Carr) can be attached onto the diaphragm of the speaker (diameter: 21 cm). The airway bioreactor can be loaded on the plate during mechanical oscillation. The frequency and amplitude of the shaker are manipulated by feeding a computer-generated sinusoidal waveform to the shaker. The response of the shaker is measured using an accelerometer (IIS3DWBTR, STMicroelectronics) or a high frame-rate camera (e.g., 240 frames per second; iPhone 11 Pro).

Further embodiments and details relating to the present invention can be found in the in publications entitled “Imaging-Guided Bioreactor for De-Epithelialization and Long-Term Cultivation of Ex Vivo Rat Trachea”, “Homogeneous Distribution of Exogeneous Cells onto De-Epithelialized Rat Trachea via Instillation of Cell-Loaded Hydrogel”, and “Imaging-Guided Bioreactor for Generating Bioengineered Airway Tissue,” the entire contents of all of which are incorporated herein by reference and made a part of the present application for all purposes.

It will be understood that the embodiments described herein are merely exemplary and that a person skilled in the art may make many variations and modifications without departing from the spirit and scope of the invention. All such variations and modifications are intended to be included within the scope of the invention.

1. Apparatus for real-time in situ tissue imaging, comprising:
a tissue culture chamber adapted to hold a tissue of interest;
a culture medium connected to said tissue culture chamber;
a flow control module configured to act on the tissue of interest;
a pressure control module configured to act on the tissue of interest;
a force control module configured to act on the tissue of interest; and
an imaging module adapted to image the tissue of interest in said tissue culture chamber.

2. The apparatus of claim 1, further comprising a plurality of sensors and actuators adapted to control a local tissue environment of the tissue of interest.

3. The apparatus of claim 2, wherein the local tissue environment is configured to imitate physiological microenvironments.

4. The apparatus of claim 1, wherein the tissue of interest is a segment of human or animal lung tissue.

5. The apparatus of claim 1, wherein said imaging module is adapted for visualization and continuous monitoring in a non-destructive way.

6. The apparatus of claim 5, wherein said imaging module is adapted to monitor the tissue of interest at a single cell level.

7. The apparatus of claim 1, wherein the tissue of interest is lung tissue and the apparatus is configured to simulate mucociliary impairment in vitro.

8. The apparatus of claim 7, wherein said imaging module is adapted to track mucociliary flow over a surface of lumen of the lung tissue by tracking microparticles.

9. The apparatus of claim 1, wherein the apparatus is adapted to simulate a breathing condition on the tissue of interest by regulation of flow rate and air flow pressure via said pressure control module and said flow control module.

10. The apparatus of claim 9, wherein the simulated breathing condition mimics coughing.

11. The apparatus of claim 1, adapted to model a disease using the tissue of interest.

12. The apparatus of claim 11, wherein the disease is a breathing disorder.

13. The apparatus of claim 1, wherein said imaging module is adapted to evaluate effects of a drug on the tissue of interest.

14. The apparatus of claim 13, further comprising a liquid instillation means adapted to deliver said drug to said tissue culture chamber.

15. The apparatus of claim 13, further comprising an aerosolization means adapted to deliver said drug to said tissue culture chamber.

16. The apparatus of claim 13, wherein the drug is a gene therapy.

17. The apparatus of claim 13, wherein said imaging module is configured to employ in situ microscopic fluorescent imaging or bioluminescent imaging.

18. The apparatus of claim 1, wherein said force control module comprises an electromagnetic shaker.

19. The apparatus of claim 1, wherein said flow control, pressure control and force control modules are adapted to replace endogenous airway cells of the tissue of interest with exogenous airway primary cells or stem cells.

20. The apparatus of claim 19, wherein said force control module operates in conjunction with a delivered chemical treatment via said flow control module to remove cellular components of the tissue of interest.

21. The apparatus of claim 19, wherein said flow control module is adapted to deliver exogenous cells for repopulation of the tissue of interest with an in-vitro cultured human airway tissue scaffold.

22. The apparatus of claim 21, wherein said scaffold is adapted for delivery via a hydrogel medium.

23. The apparatus of claim 21, wherein said scaffold is adapted for delivery via a cell culture medium.

24. The apparatus of claim 1, wherein said imaging module is adapted for monitoring removal of endogenous cells and distribution of exogenous cells in in-vitro cultured human airway tissues by using in situ microscopic fluorescent imaging modality.

25. The apparatus of claim 1, adapted to evaluate a viscous mucus-mimetic fluid deposited on the tissue of interest.

26. The apparatus of claim 1, wherein said force control module is configured to modulate frequency, amplitude and/or magnitude of an applied mechanical vibration.

27. The apparatus of claim 1, wherein said imaging module comprises an optical fiber imaging probe.

28. The apparatus of claim 27, wherein said optical fiber imaging probe has a diameter of 500 microns.

29. The apparatus of claim 1, wherein said flow, pressure and force control modules are computerized.

30. A cough generation system, comprising:
a tissue culture chamber adapted to hold a tissue of interest;
a culture medium connected to said tissue culture chamber;
a flow control module configured to act on the tissue of interest;
a pressure control module, including a computer-controlled air compressor, configured to act on the tissue of interest;
a force control module configured to act on the tissue of interest;
an imaging module adapted to image the tissue of interest in said tissue culture chamber;
a pressure sensor, operating in connection with said pressure control module;
a flow sensor, operating in connection with said flow module; and
a valve.

31. A synthetic mucus, comprising:
mucin;
low molecular weight free DNA;
a plurality of salts;
a buffer;
a lecithin source;
a source of amino acids; and
xanthan gum.

32. The synthetic mucus of claim 31, further comprising antibiotics.
